(19) United States
(12) Patent Application Publication
(10) Pub. No.: US 2026/0216489 A1
(43) Pub. Date: Jul. 30, 2026

(54) INTRAVASCULAR ACCESS DEVICE

(71) Applicant: VenoCare, Inc., Doral, FL (US)

(72) Inventors: Raul LEYTE-VIDAL, Doral, FL (US); Amir BELSON, Savyon (IL); Ryan PATTERSON, Kaysville, UT (US); Daniel PICO, Doral, FL (US)

(21) Appl. No.: 19/143,890

(22) PCT Filed: Dec. 27, 2023
(86) PCT No.: PCT/US2023/086107

§ 371 Date: Jun. 26, 2025

Related U.S. Application Data
(60) Provisional application No. 63/477,378, filed on Dec. 27, 2022.

(51) Int. Cl. A61M 39/02; A61M 25/00; A61M 25/01; A61M 25/06; A61M 25/09

(52) U.S. Cl. CPC A61M 39/0247; A61M 25/0097; A61M 25/0113; A61M 25/0606; A61M 2025/0008; A61M 25/09; A61M 2039/0258; A61M 2039/0291

(57) ABSTRACT

An intravascular access device having a handle with a slot through the handle surface and a plurality of ratchet teeth aligned on the interior of the handle. A slider positioned in the slot with a slider leg that extends to engage the ratchet teeth. A needle carrier positioned in the handle with a proximal dock configured to receive the slider leg and a needle coupled to the distal end. The needle has a longitudinal flat surface clocked along a length of the needle body and extends through a catheter and catheter hub releasably coupled to the distal end of the handle. A guide element that extends through the slider leg, through a needle carrier channel, and along a flat surface of the needle within the catheter. The slider being configured to distally advance the guide element from the catheter distal end as the slider is distally advanced along the ratchet teeth and within the handle slot.

### CROSS REFERENCE TO RELATED APPLICATIONS

This patent application claims priority to U.S. Provisional Patent Application No. 63/477,378, filed Dec. 27, 2022, titled “INTRAVASCULAR ACCESS DEVICE,” which is incorporated by reference in its entirety.

### INCORPORATION BY REFERENCE

All publications and patent applications mentioned in this specification are herein incorporated by reference to the same extent as if each individual publication or patent application was specifically and individually indicated to be incorporated by reference.

### FIELD

The present invention relates generally to methods and systems for performing vascular access. More particularly, the present invention provides a catheter and needle assembly with an integrated guide element or structure for transcutaneous insertion of the catheter into a patient's arterial or venous vasculature.

### BACKGROUND

One common type of vascular access is called venipuncture. Venipuncture refers generally to the process of obtaining intravenous access for any one of a variety of purposes, including intravenous infusion, therapy, blood sampling, and the like. In the hospital, for example, venipuncture is commonly used to place a small intravenous catheter for delivering intravenous fluids, drug delivery, blood sampling and the like.

While venipuncture and other forms of vascular access in relatively healthy patients can be a simple matter, such access is often needed in patients who are not healthy and may have small, tortuous, collapsed, fragile, and/or difficult to locate arteries and/or veins. In such patients, venipuncture and other forms of vascular access can be very challenging, particularly to less experienced phlebotomists, paramedics, nurses, and other health care practitioners.

In addition to difficult access, many vascular catheter placement systems can result in accidental punctures and/or accidental needle contamination during or after placement of the intravascular catheter. Still further, some conventional catheter placement devices employ relatively complex deployment handle movements that lead to increases both cost and complexity. Additionally, conventional handle placement and movements can obscure the presence and status of the needle and guide structure or guide element components of the tool, thus making use of the insertion tool less intuitive.

Recent developments in intravascular access devices (IVADs) provide for dramatic improvement to previous devices and methods. However, the present invention provides novel solutions and substantial improvements to the operation, adaptability, safety, application, etc.

For these reasons, it would be desirable to provide improved methods, systems, and tools for intravascular access using needles and guide structures. It would be particularly desirable to provide intravascular access deployment systems and assemblies having operational feedback mechanisms, step-processes for storage, deployment and adjustment and, even more desirably, to provide components which increase efficacy and improve safety during use. At least some of these objectives will be met by the various embodiments that follow.

### SUMMARY OF THE DISCLOSURE

In one aspect, there is provided an intravascular access device having a handle having a proximal end and a distal end, the distal end adapted to be releasably coupled to a proximal end of an intravascular catheter; a slot in a surface of the handle, the slot having a distal end and a proximal end; a plurality of ratchets along an interior wall of the handle; a slider extending through and configured to translate along the slot; a slider leg extending from the slider into an interior portion of the handle, the slider leg having a slider foot configured to engage and advance along the plurality of ratchets as the slider advances along the slot; a needle carrier having a distal end and a proximal end, the needle carrier disposed within the interior portion of the handle, the needle carrier distal end is coupled to an access needle and the needle carrier proximal end has a carrier dock sized and configured to receive a portion of the slider; and a guide element coupled to a portion of the slider within the interior portion of the handle, the guide element extending along the needle carrier, exiting the interior portion of the handle and extending beyond the distal end of the handle. In one aspect, there is a slider protrusion on the slider leg adapted and configured to engage with a dock opening in the carrier dock. Additionally, when the slider is in the distal end of the slot a slider protrusion on the slider leg is engaged with a dock opening in the carrier dock. There may also be a slider knee on the slider leg configured to flex in response to the movement of the slider foot along the plurality of ratchets. Additionally or optionally, a proximal ratchet trough within the interior of the handle and proximal of the most proximal ratchet of the plurality of ratchets. There may be an instance when the slider is in a proximal end portion of the slot, the slider foot is in the proximal ratchet trough, or optionally the plurality of ratchets are evenly spaced along the interior wall of the handle. In another version, a proximal space and a distal space are within the plurality of ratchets dividing the plurality of ratchets into a proximal set of ratchet teeth, a medial set of ratchet teeth and a distal set of ratchet teeth. There may also be visual markings or tactile markings or combination markings on the handle surface corresponding to any of the position of the slider relative to a proximal space, a distal space are within the plurality of ratchets, a proximal set of ratchet teeth, a medial set of ratchet teeth and a distal set of ratchet teeth.

In some configurations, when the slider is positioned to place the slider foot in the proximal ratchet trough the guide element is adjacent to the needle and a guide element tip is seated in a notched catheter tip. In some embodiments, when the slider is positioned to place the slider foot in the proximal space the guide element tip is spaced apart from the notched catheter tip or, alternatively, when the slider is positioned to place the slider foot in the distal space the guide element tip is spaced apart from a distal most end of the needle.

In an alternative embodiment, there is an intravascular access device with a handle having a proximal end and a distal end, the distal end adapted to be releasably coupled to a proximal end of an intravascular catheter; a slot in a surface of the handle, the slot having a distal end and a proximal end; a slider extending through and configured to translate along the slot; a slider leg extending from the slider into an interior portion of the handle, a needle carrier having a distal end and a proximal end, the needle carrier disposed within the interior portion of the handle, the needle carrier distal end is coupled to an access needle and the needle carrier proximal end has a carrier dock sized and configured to receive a portion of the slider; and a guide element coupled to a portion of the slider within the interior portion of the handle, the guide element extending along a channel the needle carrier, exiting the interior portion of the handle and extending beyond the distal end of the handle, the guide element comprising a flat side that corresponds with and is positioned alongside a flat side of the access needle, wherein the guide element is coupled to a portion of the slider that aligns directly with the location of the flat side of the needle.

In additional variations, there may be a plurality of ratchets along an interior wall of the handle and a slider foot on the slider leg configured to engage and advance along the plurality of ratchets as the slider advances along the slot, or a slider protrusion on the slider leg adapted and configured to engage with a dock opening in the carrier dock, or when the slider is in the distal end of the slot a slider protrusion on the slider leg is engaged with a dock opening in the carrier dock. There may also be variations with a slider knee on the slider leg configured to flex in response to the movement of the slider foot along the plurality of ratchets. There may also be a proximal ratchet trough within the interior of the handle and proximal of the most proximal ratchet of the plurality of ratchets. Optionally, when the slider is in a proximal end portion of the slot, the slider foot is in the proximal ratchet trough. In some variations, the plurality of ratchets are evenly spaced along the interior wall of the handle, or a proximal space and a distal space are within the plurality of ratchets dividing the plurality of ratchets into a proximal set of ratchet teeth, a medial set of ratchet teeth and a distal set of ratchet teeth. In still other aspects, visual markings or tactile markings or combination markings on the handle surface corresponding to any of the position of the slider relative to a proximal space, a distal space are within the plurality of ratchets, a proximal set of ratchet teeth, a medial set of ratchet teeth and a distal set of ratchet teeth. Still further, when the slider is positioned to place the slider foot in the proximal ratchet trough the guide element is adjacent to the needle and a guide element tip is seated in a notched catheter tip, or, when the slider is positioned to place the slider foot in the proximal space the guide element tip is spaced apart from the notched catheter tip, or, when the slider is positioned to place the slider foot in the distal space the guide element tip is spaced apart from a distal most end of the needle.

In another alternative embodiment, there is an intravascular access device with a handle having a proximal end and a distal end, the distal end adapted to be releasably coupled to a proximal end of an intravascular catheter; a slot in a surface of the handle, the slot having a distal end and a proximal end; a slider extending through and configured to translate along the slot; a slider leg extending from the slider into an interior portion of the handle, a needle carrier having a distal end and a proximal end, the needle carrier disposed within the interior portion of the handle, the needle carrier distal end is coupled to an access needle and the needle carrier proximal end has a carrier dock sized and configured to receive a portion of the slider; and a guide element coupled to a portion of the slider within the interior portion of the handle, the guide element extending along a channel the needle carrier, exiting the interior portion of the handle and extending beyond the distal end of the handle, the guide element comprising a flat side that corresponds with and is positioned alongside a flat side of the access needle, wherein the guide element comprises a transition segment from a top portion of the slider to the location of the flat side of the needle.

In some variations, the guide element transition segment couples the slider to a needle flat side located on a right side portion of the handle, a needle flat side located on a left side portion of the handle, or a needle flat side located on a bottom portion of the handle. In another aspect, a plurality of ratchets along an interior wall of the handle and a slider foot on the slider leg configured to engage and advance along the plurality of ratchets as the slider advances along the slot. Optionally, there may also be a slider protrusion on the slider leg adapted and configured to engage with a dock opening in the carrier dock. In one variation, when the slider is in the distal end of the slot a slider protrusion on the slider leg is engaged with a dock opening in the carrier dock. There may also be a slider knee on the slider leg configured to flex in response to the movement of the slider foot along the plurality of ratchets or a proximal ratchet trough within the interior of the handle and proximal of the most proximal ratchet of the plurality of ratchets. In one variation, when the slider is in a proximal end portion of the slot, the slider foot is in the proximal ratchet trough. There are embodiments where the plurality of ratchets are evenly spaced along the interior wall of the handle. In one aspect, a proximal space and a distal space are within the plurality of ratchets dividing the plurality of ratchets into a proximal set of ratchet teeth, a medial set of ratchet teeth and a distal set of ratchet teeth. In some embodiments, there are visual markings or tactile markings or combination markings on the handle surface corresponding to any of the position of the slider relative to a proximal space, a distal space are within the plurality of ratchets, a proximal set of ratchet teeth, a medial set of ratchet teeth and a distal set of ratchet teeth. Optionally, when the slider is positioned to place the slider foot in the proximal ratchet trough the guide element is adjacent to the needle and a guide element tip is seated in a notched catheter tip or when the slider is positioned to place the slider foot in the proximal space the guide element tip is spaced apart from the notched catheter tip or when the slider is positioned to place the slider foot in the distal space the guide element tip is spaced apart from a distal most end of the needle.

In one variation, there is also provided with the intravascular device above an intravascular catheter having a lumen configured to accommodate the access needle extending therethrough and a notched distal end portion sized and configured to receive the guide element tip.

In still another variation, there is a cover for a vascular access device having a body having a closed distal end and an open proximal end defining an interior portion, the open proximal end sized and configured to releasably couple to a distal end of a handle of the vascular access device; a pair of alignment features within the interior portion defining a lumen extending from the open proximal end to a recess in a distal most portion of the interior portion; and a ramp along the interior portion proximal to recess. In one configuration, there is an intravascular access device coupled to the open proximal end of the cover. Still further, the cover may be adapted and configured for use with an intravascular access device having a handle having a proximal end and a distal end, the distal end adapted to be releasably coupled to a proximal end of an intravascular catheter; a slot in a surface of the handle, the slot having a distal end and a proximal end; a slider extending through and configured to translate along the slot; a needle carrier having a distal end and a proximal end, the needle carrier disposed within the interior portion of the handle, the needle carrier distal end is coupled to an access needle and the needle carrier proximal end has a carrier dock sized and configured to receive a portion of the slider; and a guide element coupled to a portion of the slider within the interior portion of the handle, the guide element extending along the needle carrier, exiting the interior portion of the handle and extending beyond the distal end of the handle, wherein the access needle extends along and between the alignment features and a tissue penetrating tip of the needle is proximal to the ramp, further wherein the slider is positioned along the slot such that a distal tip of the guide element is distal to the ramp and within the recess. In one variation, proximal movement of the slider moves the distal end of the guide element over the ramp and into a notched distal end portion of the catheter lumen. Still further, the handle further comprises a visual or a tactile marking to indicate the position of the distal end of the guide element relative to the notched distal end of the catheter or the tissue penetrating tip of the needle. In additional variations, the cover is adapted and configured for use with any of the vascular access devices described herein.

In yet another embodiment, there is provided a method of accessing a blood vessel of a patient by advancing a tissue penetrating distal tip of an access needle of an intravascular access device in an armed configuration into a blood vessel of the patient; engaging a guide element with an leg of a slider, the guide element extending through a proximal end of the intravascular access device through a distal tip of a catheter; advancing the slider distally within a slot that extends longitudinally along the intravascular access device, wherein the slider leg is advanced across a ratchet system on an interior surface of the intravascular access device; advancing a distal tip of the guide element from within a notch at the distal tip of the catheter into blood vessel distal to the tissue penetrating tip of the access needle;

advancing a distal portion of the catheter hub in the blood vessel along the guide structure; and retracting a needle carrier coupled to a proximal end of the access needle into the intravascular access device. In other steps, the is a method of applying a force to the slider before advancing the slider, wherein the force increases a frictional engagement between the leg of the slider and the guide element. Optionally, there is a step of confirming vascular access when blood is observed in a catheter hub coupled to the distal end of the intravascular access handle. Additionally, there is a step of uncoupling a catheter hub from a distal end of the intravascular access device handle after the catheter is positioned within the blood vessel. In other variations, there is a step of withdrawing the intravascular access device handle proximally along the guide element after the slider is advanced to a distal end of the slot. In one aspect, there is a step of determining a length of guidewire advanced into the blood vessel based on the distance the slider was advanced over the ratchet system. Optionally, the slider is advanced from a neutral configuration with the leg of the slider contacting a proximal trough of the ratchet system. In another variation, the ratchet system preventing proximal translation of the slider after the slider has been advanced, or advancing the slider leg to contact a proximal end of the needle carrier. In some embodiments there is a process of ceasing to perform the step of advancing a tissue penetrating distal tip of an access needle of the intravascular catheter assembly when bleed back is observed in the intravascular catheter assembly.

In still other configurations, there is provided an intravascular access device as described above adapted for use with a needle cover having a proximal opening sized to engage with a portion of the handle; a lumen in communication with the proximal opening, the lumen sized to receive the needle and catheter; a ramp at a distal position of the lumen adjacent to a recess in the distal most portion of the lumen wherein in use with an IVAD within the needle cover the distal tip of the needle is separated from the guide element by a portion of the ramp and the guide element is in a coiled condition within the recess. In some configurations, the needle cover and the intravascular device are adapted and configured to interoperate into one or more of a neutral condition, an armed condition, a ready for use condition and a disposal condition. There may also be versions of the methods above with one or more steps of interaction between the vascular access device and a needle cover so as to render the vascular access device into one or more of a neutral condition, an armed condition, a ready for use condition and a disposal condition.

In still another variation, there is an intravascular access device having a handle comprising a proximal end and a distal end, a slot in a surface of the handle having a distal end and a proximal end, and a plurality of ratchet teeth along an interior of the handle; a slider positioned in the slot, the slider comprising a slider leg extending towards the plurality of ratchet teeth; a needle carrier having a distal end coupled to an access needle and a proximal end positioned within the handle; a catheter having a proximal end coupled to a catheter hub, the catheter hub configured to engage the distal end of the handle, the catheter comprising a lumen configured to accommodate the access needle extending therethrough; a guide element extending from the slider through the catheter lumen, the guide element comprising a distal tip configured to engage a notched distal tip of the catheter, wherein the guide element is configured to be advanced distally from the notched distal tip of the catheter by the slider leg. In some variations, there may be a dock at the proximal end of the needle carrier adapted to receive the slider leg therein, or a channel along the needle carrier adapted to receive the guide element, or a channel adapted to receive the guide element therethrough extends along the needle carrier. Still other variations the access needle comprises a flat surface along the needle exterior or the needle carrier comprises a channel along the needle carrier, the channel adapted to receive the guide element therethrough or the channel is positioned on the needle carrier at the same clock position as the needle flat surface. In still other variations there may be one or more of a ratchet system is configured to prevent the slider from retracting after the slider has been advanced distally along the ratchet system, or a slider further comprising one or more support element configured to engage a perimeter of the slot to prevent rotational displacement of the slider, or where the ratchet system comprises a plurality of ratchet teeth positioned distal to a trough near a proximal end of the handle. In some other variations, an aperture locatable on the slider leg, wherein the guide element extends through the aperture and the aperture is configured to an exterior of the guide element when the slider is advanced along the slot or a guide element channel extending longitudinally along an exterior of the needle carrier, the guide element channel configured to route the guide element therethrough. In some aspects, there may also be a guide element extends through the catheter lumen along an exterior surface of the needle or configurations when the slider is in a distal most position a distal portion of the guide element transitions to an atraumatic tip beyond the distal tip of the needle.

In still other additional configurations of the vascular access device, the slider is configured to transition from a neutral configuration to an advanced configuration as the slider is advanced distally from a proximal trough region of the ratchet system or the slider leg is spring biased towards the ratchet system or the distal end of the needle, distal tip of the guide element and distal end of the catheter are configured to dilate an opening into a vessel. There may also be a guide element notch and a guide element distal tip having a proximal end and a distal end, wherein the proximal end of the guide element distal tip is configured to engage the guide element notch or a bevel surface of the needle distal end is coplanar with the guide element distal tip and the catheter distal end. In other aspects, there is an actuation element operably coupled to the needle carrier, the actuation element configured to displace the needle carrier proximally within the handle when an actuator trigger is engaged or a slider leg dock at a proximal end of the needle carrier, the slider leg dock configured to receive a portion of the slider leg therein. In some embodiments, the slider is configured to engage the proximal end of the needle carrier. Other embodiments provide a ratchet type system configured to provide feedback to a user of the position of the guide element distal tip within a vessel.

In still other variations of the vascular access device, the device is configured to transition from an armed configuration, wherein the slider leg is positioned adjacent to a proximal stop of the ratchet system to an advanced configuration wherein the slider leg is advanced distal to one or more ratchet teeth of the ratchet system. In one aspect, the distal tip of the guide element is nested with the guide element notch in the armed position. In another variation, the ratchet type system comprises a plurality of ratchet teeth along a length of the ratchet system and parallel to the slot. In some embodiments, the guide element is made entirely or partially of a polymeric material, a PTFE, a Nylon. In some configurations, the guide element is made entirely or partially of metal, comprising Nitinol, comprising Elgiloy or similar materials. In one additional aspect, the catheter is configured for use as an introducer, dilator, catheter component of an infusion set, a catheter component in a blood collection set, a catheter component in a safety winged blood collection set, or a catheter component in an intravenous catheter.

### BRIEF DESCRIPTION OF THE DRAWINGS

The novel features of the invention are set forth with particularity in the claims that follow. A better understanding of the features and advantages of the present invention will be obtained by reference to the following detailed description that sets forth illustrative embodiments, in which the principles of the invention are utilized, and the accompanying drawings of which:

FIG. 1 is a perspective view of an exemplary intravascular access device in a deployed configuration with a guide element advanced beyond a needle tip.

FIG. 2 is a perspective view of the intravascular access device shown in FIG. 1 with internal elements visible through a transparent handle.

FIG. 3A is a side elevation view of the intravascular access device shown in FIG. 1 with the handle and catheter hub removed to expose a configuration of a needle carrier, slider and guide element positioning.

FIG. 3B is a side elevation view of an intravascular access device as shown in FIG. 1 with the handle and catheter hub removed to expose a configuration of a needle carrier, slider and guide element routing through an alternative clocked position along the needle carrier and needle.

FIG. 3C is a side elevation view of an intravascular access device as shown in FIG. 1 with the handle and catheter hub removed to expose a configuration of a needle carrier, slider and guide element routing through an alternative clocked position along the needle carrier and needle.

FIG. 4 is a side elevation view detailing a medial region of the intravascular access device shown in FIG. 2 including an example of a ratchet system, as described herein.

FIG. 5A is a cross-section from a distal perspective view of the intravascular access device shown in FIG. 2 with details of the slider engagement with the ratchet teeth.

FIG. 5B is a lateral cross section view of the intravascular access device shown in FIG. 2 from a proximal perspective illustrating additional details of the slider engagement with the ratchet teeth.

FIG. 6A is a top plan view of the distal section of the intravascular access device shown in FIG. 2 with the slider in a distally advanced position of the handle.

FIG. 6B is a close up view of the intravascular access device in FIG. 6A with additional details of a catheter hub configuration including the guide element path from the needle carrier through a hemostasis valve in the catheter hub.

FIG. 7 is a perspective view of the intravascular access device shown in FIG. 1 with the handle and catheter hub removed to expose the needle carrier, actuation button and hemostasis valve positioned at a proximal end of the catheter.

FIG. 8 is a distal perspective view from the top of the intravascular access device shown in FIG. 1 with the handle, catheter hub and actuation button removed to expose details of the guide element pathway from the slider to the catheter.

FIG. 9 is a distal perspective view of the intravascular access device shown in FIG. 1 with the catheter hub removed to expose details of the the distal end of the handle and guide element extending from the needle carrier to a space between a flat surface of a needle and an interior of the catheter.

FIG. 10A to FIG. 10D are cross section views of exemplary needle configurations including a flat surface along the needle body that may be positioned around the needle exterior surface.

FIG. 11 is perspective view of a distal segment of a catheter, needle and guide element couplable to the handle of an intravascular access device such as the device shown in FIG. 1.

FIG. 12A is a perspective view of a slider from an intravascular access device as shown in FIG. 1 from the distal end.

FIG. 12B is a perspective view from the proximal end of the slider from FIG. 12A.

FIG. 12C is a perspective view from the distal end of the slider with alternative guide element lumen positions from FIG. 12A.

FIG. 13 is a view of a proximal region of the slider shown in FIG. 12A with additional details of alignment features and a guide element lumen positioned in the slider extension.

FIG. 14 is a profile view of a cross-section from the distal region of the intravascular access device shown in FIG. 2.

FIG. 15 is a proximal perspective view of the intravascular access device shown in FIG. 2 with additional details of the slider extension engaging the ratchet teeth in a proximal most position while in a ready-to-use configuration.

FIG. 16 is a proximal perspective view of the intravascular access device shown in FIG. 2 zoomed in to the proximal end of the needle carrier with slider adjacent to the needle carrier dock as the slider is advanced distally along the handle.

FIG. 17 shows a subsequent position of the slider extension distally advanced beyond the position shown in FIG. 16 with the slider extension engaging the needle carrier dock.

FIG. 18 is an additional view of the sequence from FIG. 16 and FIG. 17 where a cross section of a side elevation view shows details of the engagement between the slider extension and the needle carrier dock in a docked configuration when the slider is advanced to a distal most position in the handle.

FIG. 19A is a flow chart of an exemplary method of a process of placing a neutral condition intravascular access device (e.g., the device shown in FIG. 1) into a needle cover and transitioning a neutral condition intravascular access device into an armed condition and a ready for use condition.

FIG. 19B is a flow chart of an exemplary method of a process using an intravascular access device (e.g., the device shown in FIG. 1) in a ready for use condition to enter into a blood vessel and then advancing a guide element by advancing a slider along a series of rachet points to then guide the needle and catheter hub into a desired position within the blood vessel.

FIG. 20 is a cross section view of a needle cover.

FIG. 21 is a rear isometric view of the needle cover of FIG. 20 with a distal end of an intravascular access device (e.g., the device shown in FIG. 1) adjacent to the cover opening.

FIG. 22A and FIG. 22B are enlarged cross section views of a guide element advancing up a ramp (FIG. 22A) and then down the ramp (FIG. 22B) that separates the guide element from the needle tip and guides the guide element into a recess in the needle cover as shown in FIG. 22B.

FIG. 23 is an enlarged cross section of the intravascular access device of FIG. 22B showing the relative positions of the piercing needle tip to the ramp. The guide element is removed for clarity.

FIG. 24A and FIG. 24B are isometric detailed interior and isometric transparent views, respectively, of the intravascular access device as configured in a storage condition of FIG. 22B.

FIG. 25 is a cross section view showing the movement of the guide element along the ramp and into position against the catheter tip during the transition into an armed condition.

FIGS. 26A and 26B are isometric detailed interior and isometric transparent views of an intravascular access device in an armed condition where the guide element has been transitioned into position as shown in FIG. 25. FIG. 26B illustrates the direction of the slider to accomplish the guide element movement shown in FIG. 25.

FIG. 27 is a perspective view of the distal end of an armed intravascular access device as shown in FIG. 26A and FIG. 26B with the needle cover removed as shown in FIG. 21 to transition the intravascular access device into a ready to use condition.

FIG. 28 is an isometric view of an intravascular access device in a neutral condition as in FIG. 24A and FIG. 24B having a picogram affixed to the needle cover and the slider indicating the order of movements to transition the intravascular access device from a neutral or storage condition to a ready to use condition.

FIG. 29 is a detailed view of the distal region of an intravascular access device with an alternative ratchet teeth configuration including deployment length identification features.

FIG. 30A to FIG. 30C are perspective views of intravascular access device handles including exterior features adapted to indicate the length of guide element deployment corresponding to the slider position in the handle slot.

### DETAILED DESCRIPTION

An intravascular access device (IVAD) as described herein can comprise elements configured to access the vasculature of a patient in a safe, efficient and effective manner with intuitive manipulation of a needle controlled by a slider operably couples thereto. The slider can be operably coupled to the IVAD and engaged by a user with positive feedback to indicate a position of the slider relative to an IVAD handle (e.g., body) allowing the user to maintain constant focus on the placement and advancement of a needle, catheter, or other element into a blood vessel. The IVAD may have one or more features to facilitate the static and/or dynamic positioning of the slider and elements operably couped thereto relative to the IVAD and/or the patient.

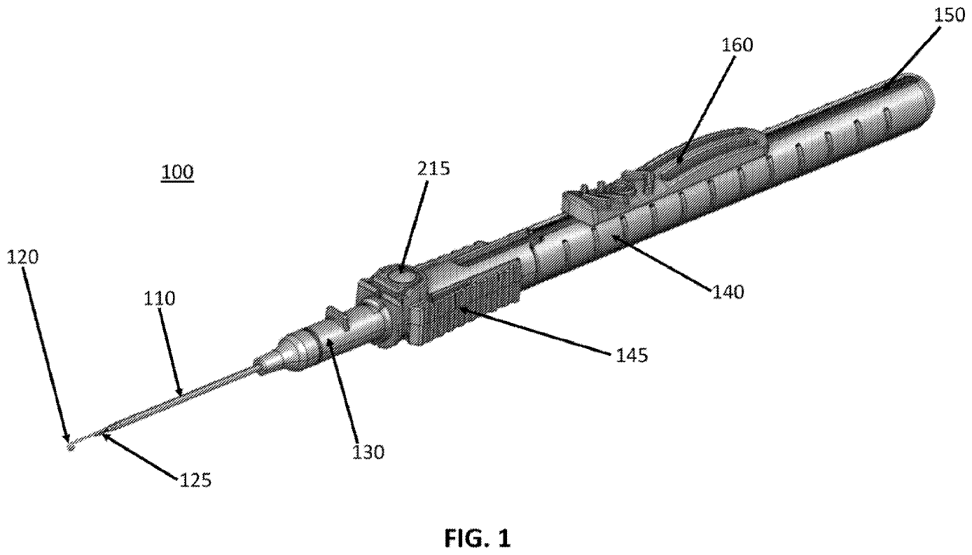

FIG. 1 is a perspective view of an embodiment of an IVAD 100 having a catheter 110, guide element (e.g., guidewire) 120 and needle 125 operably coupled to a distal end of the IVAD 100. As shown here, the guide element 120 is extended beyond the tissue penetrating tip of the needle 125 with an atraumatic tip configuration as may be established when the guide wire 120 is advanced within a patient's vasculature. The catheter hub 130 is coupled to the distal end of the handle (e.g., body, housing, etc.) 140 with an access needle 125 in place within the catheter lumen (not shown).

The handle or housing 140 has a proximal end and a distal end. In some examples, the IVAD may have one or more engagement elements 145 coupled to or integrated with the handle 140. An example of the engagement element 145 provides a surface and geometry configured to promote positive interaction between the user and the housing 140 during use. For example, the engagement element 145 tapers proximally along a length of the housing 140 where a user may manipulate the IVAD during use without having to increase a gripping strength on the handle 140. A slot 150 runs along a length of the housing 140 providing access for the slider 160 to engage with the guide element 120 extending through an interior of the handle 140. The slider 160 can be positionable along a length of the slot 150. Positioning of the slider 160 within the slot 150 can be facilitated by a ratcheting system comprising corresponding elements of the slider and IVAD housing 140. The catheter 110 has a distal end and a proximal end and an interior lumen along a length of the catheter 110. The catheter proximal end can be adapted and configured to engage with the distal end of the handle 140. The catheter proximal end may also be configured to engage with one or more connections, hubs, peripheral devices, etc. once inserted into a vessel and the handle 140 is removed. For example, the catheter proximal end may comprise features to engage with a luer lock, fluid coupling or other suitable medical connector or fitting.

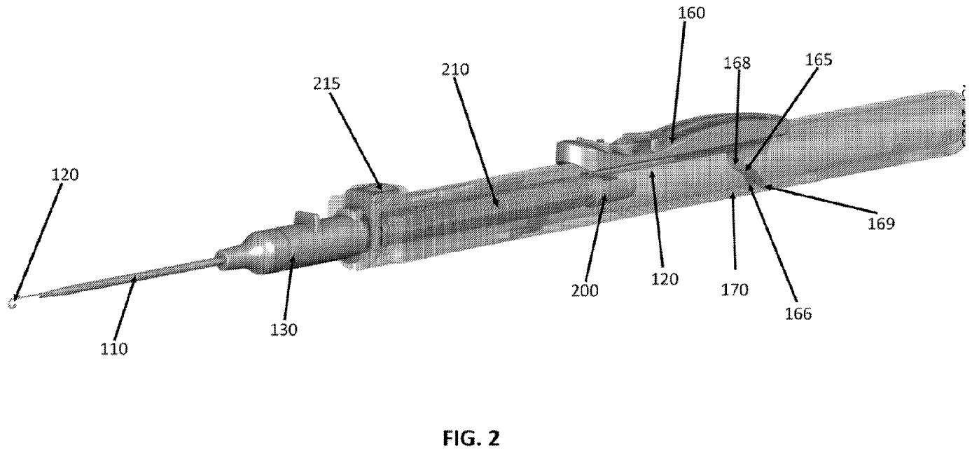

FIG. 2 is a perspective view of an IVAD 100 in a similar configuration to the examples shown in FIG. 1. Here, the housing 140 is transparent. The slider 160 comprises a slider leg 165 extending from the slider 160 into an interior of the housing 140. The slider leg 165 may extend to contact an interior surface of the housing 140. A ratcheting system may comprise one or more elements of the slider 160 and corresponding elements locatable on an interior of the handle 140. As illustrated in FIG. 2, a ratcheting system may include the slider leg 165 and one or more elements of a non-uniform interior surface of the handle. For example, ratchet teeth 170 are positioned on an interior surface of the handle and oriented in a similar direction as the slot 150 and/or direction of slider 160 translation from a proximal position to a distal position in the slot 150.

An actuation element 210 is visible in FIG. 2 and locatable near a distal end of the IVAD housing. The illustrated example of an actuation element 210 is a spring coiled around the needle carrier 200 and positioned around an exterior surface of the needle carrier 200 within the handle. The actuation element 210 can be configured to bias the needle carrier 200 to a proximal position and may be selectively activated (e.g., released by an actuator trigger 215). In some examples, the needle carrier may be retained by one or more grooves, channels, or any style of retention mechanism in operable communication with the actuator trigger 215 such that the trigger may be selectively engaged to release the needle carrier 200 allowing the actuation element 210 to force the needle carrier 200 primally within the handle. In some examples, the needle carrier 200 is operably coupled to a proximal end of the needle and is configured to draw, pull, or otherwise transition the needle into the handle interior.

In some examples, an IVAD described herein may be described in different configurations based on a stage of use (e.g., position of one or more elements of the IVAD) Some examples of IVAD configurations may include a ready-to-use configuration, storage configuration, armed configuration, locked configuration, deployed configuration, etc. For example, in a ready-to-use configuration the slider can be positioned near the proximal end of the handle and in operable communication with the guide element near the proximal end of the handle. From that configuration, the IVAD may transition to a deployed configuration whereby the slider can be advanced distally within the slot, thereby advancing the guide element along a route through the intravascular access device as the guide element is advanced through the needle carrier such that a distal tip (e.g., atraumatic tip) of the guide element is advanced beyond the tissue penetrating tip of the needle. Another transition may be from a deployed configuration to a retracted configuration whereby after the catheter is deployed, the actuation trigger can be depressed to release the actuation element to displace the needle carrier and the needle into the handle.

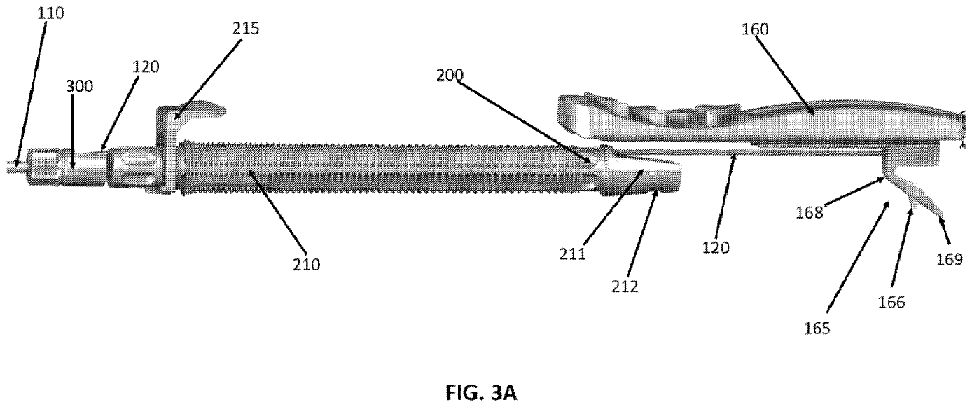

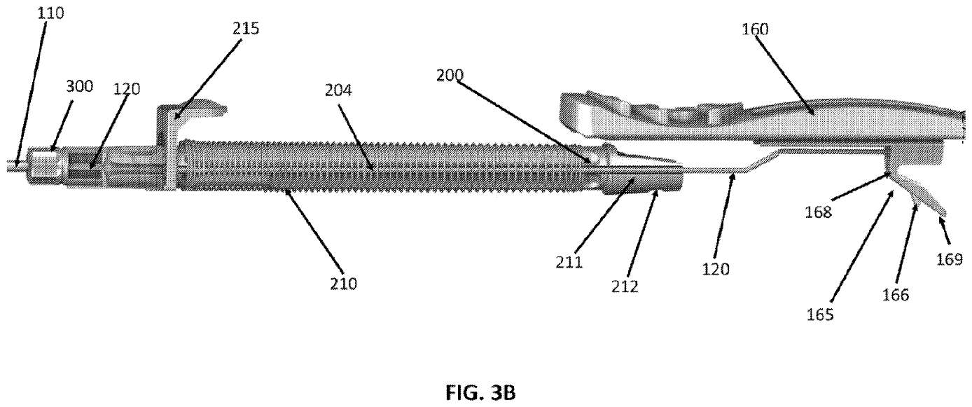

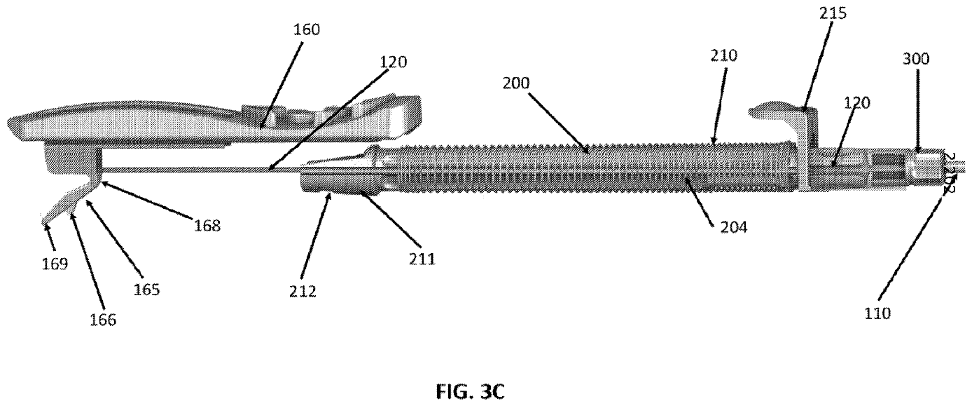

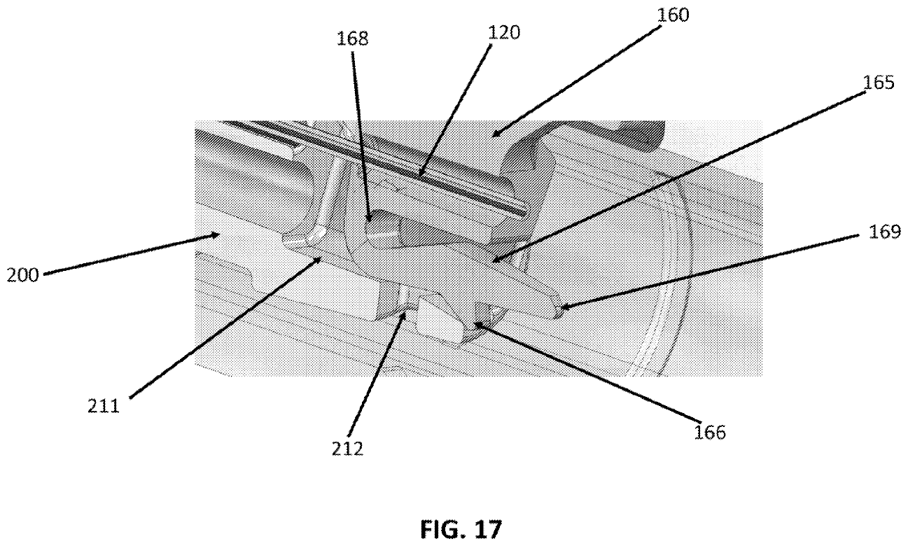

FIG. 3A is a side elevation view of an IVAD with the handle removed from view and details of the internal arrangement of elements are visible. In particular, the slider leg 165 is positioned at a proximal end of the slider 160. The slider leg 165 can extend from the slider 160 and include a slider knee 168, slider protrusion 166, and slider foot 169. The slider knee may be configured to bend or flex like a spring allowing the slider leg 165 to travel across the ratchet teeth within the handle. The slider protrusion 166 may be configured to engage the proximal end of the needle carrier 200 at the dock opening 212. The slider foot 169 at the end of the slider leg 165 is adapted to engage the plurality of ratchet teeth when the slider is advanced through the handle. For example, the slider foot 169 may engage sequential ratchet teeth during distal advancement. The foot 169 may be configured to prevent retraction of the slider 160 until the slider leg 165 has engaged the needle carrier dock 211 and the foot 169 is lifted away from the ratchet system as shown in FIG. 17. The guide element 120 is routed through the IVAD including the slider 160 and the needle carrier 200. The needle carrier can have a dock 211 near a proximal end of the needle carrier 200 including an opening 212 configured to receive the slider protrusion 166 therein, when the slider 160 is in an advanced position within the IVAD. With the handle removed from view, the actuation element 210 and actuation trigger 215 are clearly visible. The actuation trigger 215 is shown here as a button that may be selectively depressible causing the needle carrier 200 to become unlocked and propelled proximally within the handle by the actuation element 210. At a distal end or region of the IVAD, the catheter hub is removed to expose a distal end of the needler carrier 200 extending distally beyond the actuation trigger 215. A valve 300 may be positioned distally from the distal end of the handle (e.g., distal to the actuation trigger). The catheter 110 may extend through the valve 300 and the valve may be configured to regulate pressure changes as the catheter is advanced and one or more fluids (e.g., gases/liquids) are displaced from within the catheter, catheter hub, vasculature, etc.

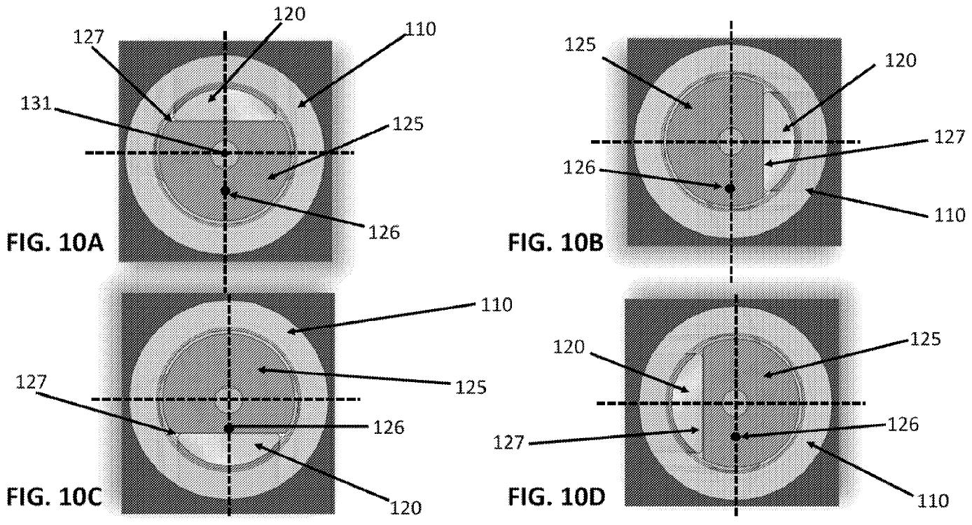

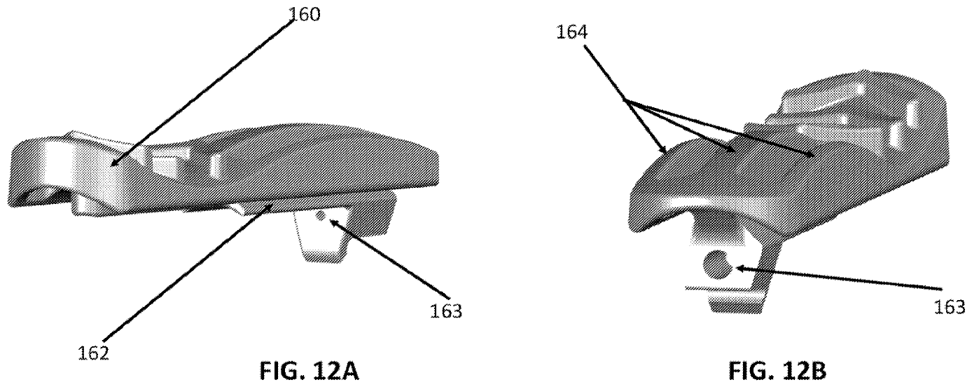

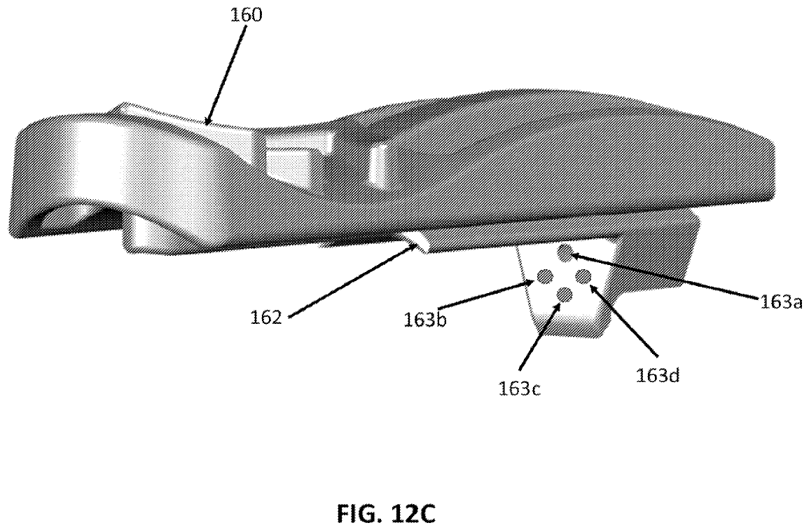

Similar to FIG. 3A, FIGS. 3B and 3C show alternative routing of the guide element 120 through the needle carrier channel 204 from the slider 160. In FIG. 3B, the guide element 120 is routes through the needle carrier 200 in the channel 204 that is clocked corresponding to the needle flat surface 127 in an orientation as shown in FIG. 10B. In this arrangement, the guide element 120 may be described as traveling through a 3 o'clock position as supported by the position of the needle carrier channel 204 and the needle flat surface 127 aligned in the same 3 o'clock position along the needle carrier 200 and needle 125 respectively. FIG. 3C illustrates another example of a different guide element routing in the 9 o'clock position with the needle carrier channel 204 along the side of the needle carrier and the needle flat surface being in the same orientation as shown in FIG. 10D. One additional and illustrative example shown in FIG. 3C is the guide element being straight or substantially straight in a linear or coaxial alignment from the slider leg 165 (e.g., the slider lumen 163) to the needle carrier channel 204. As further detailed in FIG. 12C, the slider leg 165 may comprise one or more openings positioned on the slider leg 165 to accept the guide element therethrough in a similar orientation to the clock position of the needle carrier channel 204 and the need flat surface 127.

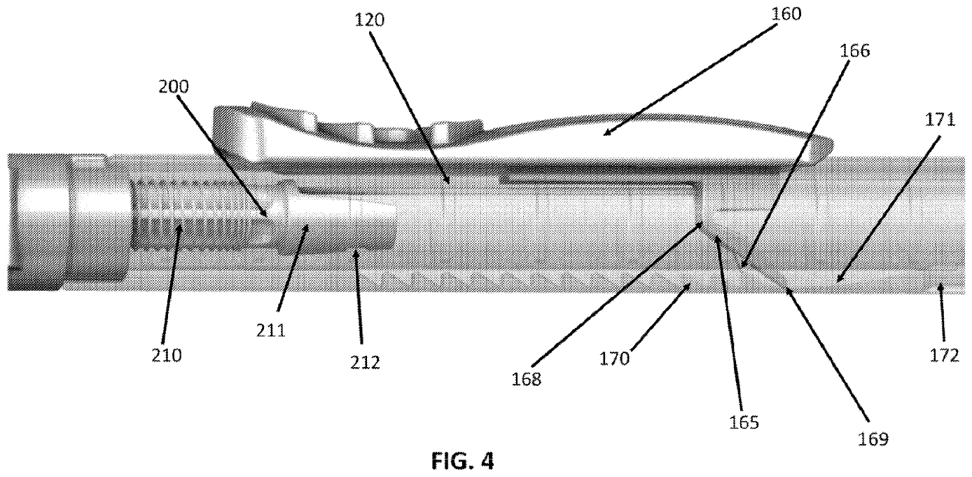

FIG. 4 shows a detailed view of a segment of an IVAD as described herein. In particular, the segment shows an example of an IVAD ratcheting system comprising the slider leg 165, slider leg protrusion 166, ratchet teeth 170, ratchet proximal trough 171, and ratchet proximal hard stop 172. From this view, the arrangement, orientation and function of the ratchet system elements is clearly visible. The slider leg protrusion 166 is shown contacting a peak or upper area of one of the ratchet teeth 170 with the terminal end of the ratchet leg positioned in the ratchet proximal trough 171. The slider may be considered to be in a neutral configuration. For example, the slider 160 may be positioned proximal to a midsection of the handle whereby the terminal end of the slider leg 165 is in the neutral zone (e.g., ratchet proximal trough 171).

In the neutral configuration illustrated in FIG. 4, the slider leg 165 position in the proximal trough 171 retains the guide element 120 to a position whereby the distal tip of the guide element may be aligned, proximal to, or sufficiently close to the tissue penetrating top of the need to prevent damage to the guide element during storage or transportation of the IVAD. In this neutral position, the slider 165 can be displaced (e.g., advanced distally) with sufficient force to overcome the engagement of the slider leg terminal end with the ratchet proximal trough 171.

The slider 160 may be distally advanced from the neutral position as illustrated in FIG. 4 and the slider leg 165 can engage the ratchet teeth 170. The distal advancement of the slider 160 through the slot 150 and along the ratchet teeth 170 may be in a sequential manner whereby contact between the slider leg 165 and ratchet teeth maintains the slider in a distally advanced position until the slider 160 is advanced further along the ratchet system or until the slider 160 (e.g., slider leg 165) engages the proximal end of the dock 211 at the proximal end of the needle carrier 200 when the needle, guide element, needle carrier, and slider are retracted proximally by the actuation element. The slider 160 engagement with the needle carrier dock 211 can be supported by the slider protrusion 166 seating within the opening 212 in the slider dock 211 to create a positive engagement between the slider and the needle carrier.

In some examples, an IVAD may transition from a neutral configuration to an armed configuration when the slider 160 is retracted proximally from the neutral position wherein the slider leg 165 is contacting the ratchet proximal trough 171 to an armed position where the slider leg 165 is contacting a ratchet proximal hard stop 172. The guide element distal end is retracted correspondingly to the slider 160 from the neutral configuration to the armed configuration. The distal end of the guide element may be retracted to engage a guide element slot (e.g., catheter notch). For example, a distal end of the catheter may comprise a notch 128 having a shape or geometry corresponding to the guide element distal tip such that the notch 128 can be configured to receive the guide element distal tip therein. Some examples of guide elements may be those described in co-pending U.S. Provisional Patent Application Ser. No. 63/408,419 entitled “GUIDE ELEMENT FOR INTRAVASCULAR ACCESS DEVICE” filed on Sep. 20, 2022, the contents of which are hereby incorporated by reference in its entirety. From the armed configuration, the slider 160 may be advanced distally (e.g., as needed) to advance the guide element 120 within the vasculature after venipuncture is complete (e.g., needle has penetrated the vessel).

The slider leg 165 may act as a spring or other resilient elements configured to be biased towards an extended position. The slider leg may be configured to provide a force (e.g., an elastic force) along a length of the slider leg 165 to the slider leg terminal end such that the contact between the slider leg (e.g., slider leg terminal end) and the interior of the handle (e.g., ratchet system) may be increased based on the spring force provided by the slider leg 165.

In some examples, the slider leg 165 may be configured to provide sufficient force to the slider leg terminal end to cause an audible or tactile feedback to a user engaging the handle as the slider is advanced over, across, through, etc. the ratcheting system. For example, as the slider 160 is advanced distally and the slider leg 165 passes over the ratchet teeth 170, an audible click may result as the slider leg terminal end contacts the trough surface after passing over a ratchet tooth. In some examples, the audible click may be a result of physical contact between the slider leg terminal end and the interior surface of the handle and may include or be accompanied by a vibration recognizable by a user engaging the handle. The click and/or vibration may provide feedback to the user that the slider has advanced to a position and may or may not be capable of retraction proximally from the current locked position. This feedback system can be calibrated or configured to provide quantifiable distances of travel for the slider 160 allowing a user to appreciate a current position or change in position (displacement) of the slider during use. For example, each click may represent or correspond to a distance such that one click may represent 1 centimeter of advancement of the slider 160 and guide element 120. In some examples, the distance of each ratchet tooth may be any predetermined distance from one another, and measurements may be provided, predetermined, or otherwise established to indicate a distance the slider has traveled; and therefore, a corresponding distance the guide element distal tip has traveled. In some examples, the distance between ratchet teeth may be less than 1 mm, equal to 1 mm, greater than 1 mm. In some examples the distance between each ratchet tooth may be the same, different, or a combination of the same distance between ratchet teeth and different distances between ratchet teeth.

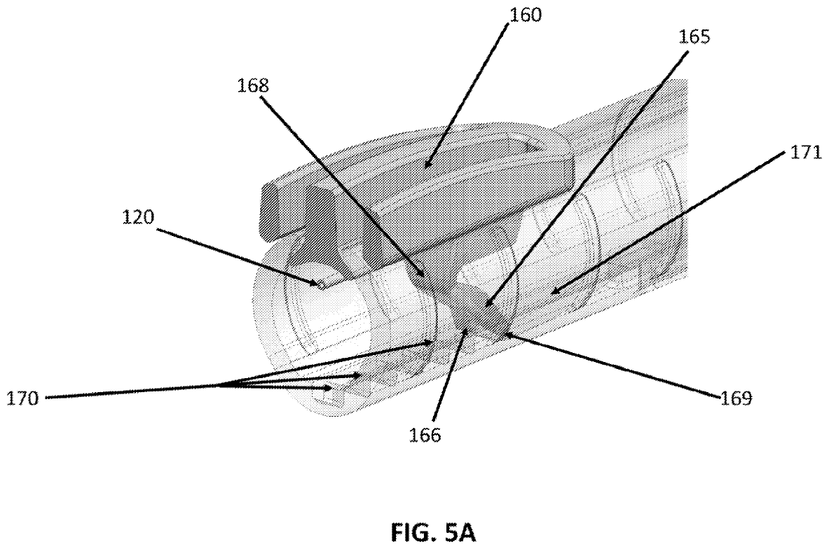

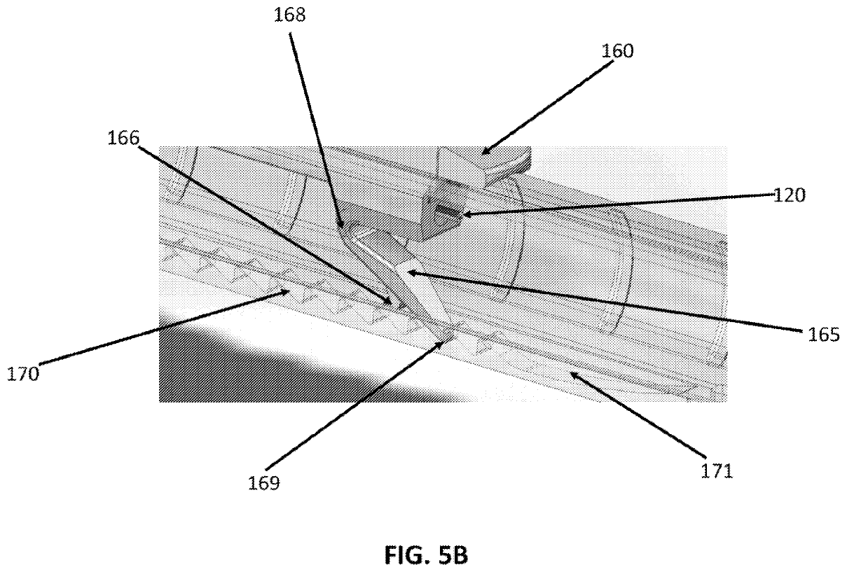

FIGS. 5A and 5B show a perspective view in cross section of the slider 160 in the neutral configuration with the slider leg 165 contacting the ratchet proximal trough 171. A cross section of guide element 120 is shown as an example of the routing arrangement from the slider 165 through the interior of the handle. Elements of the ratchet system may be more clearly visible as the ratchet teeth 170 are illustrated in line with one another and distal to the slider leg 165. In some examples, the slider 160 is advanced distally from this position and the slider leg 165 can slide over peaks of ratchet teeth 170 and settle into troughs between each of the ratchet teeth 170 as it is advanced.

The geometry of the ratchet teeth 170 may be any geometric shape. For example, any non-uniform interior surface of the housing may be considered ratchet teeth where the non-uniform surface promotes a retention of the slider as it passes over, through or across the ratchet teeth. For example, ratchet teeth may refer to an uneven surface having one or more raised portions such that the slider leg 165 may be retained or motivated to a static position in the absence of a force, between the one or more raised portions. Referring to FIG. 4, the ratchet teeth 170 are shown in a generally triangular geometry having one side with a negative slope adjacent to another side generally vertical relative to the interior surface of the handle. In this example, the slider 165 may be advanced distally such that the slider leg 165 slides over the slop and falls along the vertical side thereby preventing it from being retracted or withdrawn proximally.

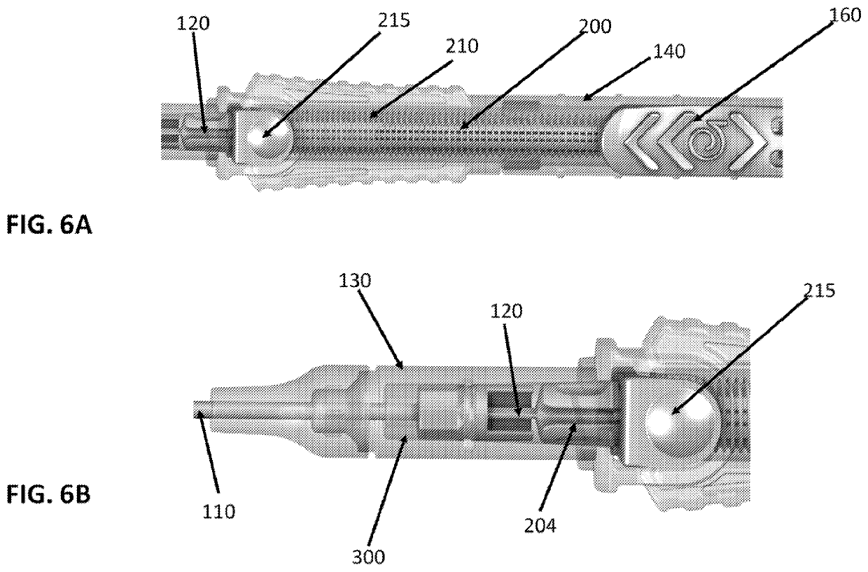

In FIG. 6A and FIG. 6B, details of an IVAD are shown in a top plan view zoomed in on the distal end of the handle 140 showing the arrangement and configuration of the handle 140, catheter hub 130, actuation element 210, actuation trigger 215 and needle carrier 200. From this view, an example arrangement of the guide element 120 is shown routed generally along the interior of the handle body and through a channel 204 in the needle carrier 200. The channel may be configured to provide stability while the guide element 120 moves within and through the handle 140.

FIG. 6B is a more detailed view of the catheter hub 130 having the valve 300 with the catheter 110 and guide element 120 generally coaxial therewith. For example, in this configuration, the guide element 120 can be advanced via the slider 160 distally into vasculature of a patient. The guide element 120 may be advanced through the needle carrier channel 204 of, through the distal end of the handle and into the catheter hub 130, where it may be further advanced through the valve 300 and an interior lumen of the catheter 110. In some examples, the guide element may be advanced through the catheter lumen and out through a distal end of the catheter (not shown) into the vasculature. In some examples, the guide element may pass through the catheter lumen having a needle extending therethrough such that the guide element may pass between an exterior surface of the needle and an interior surface of the catheter. In some examples the guide element may pass through a lumen of the needle, a lumen of the catheter, both, or a combination thereof depending on the arrangement, configuration, and type of use of the guide element.

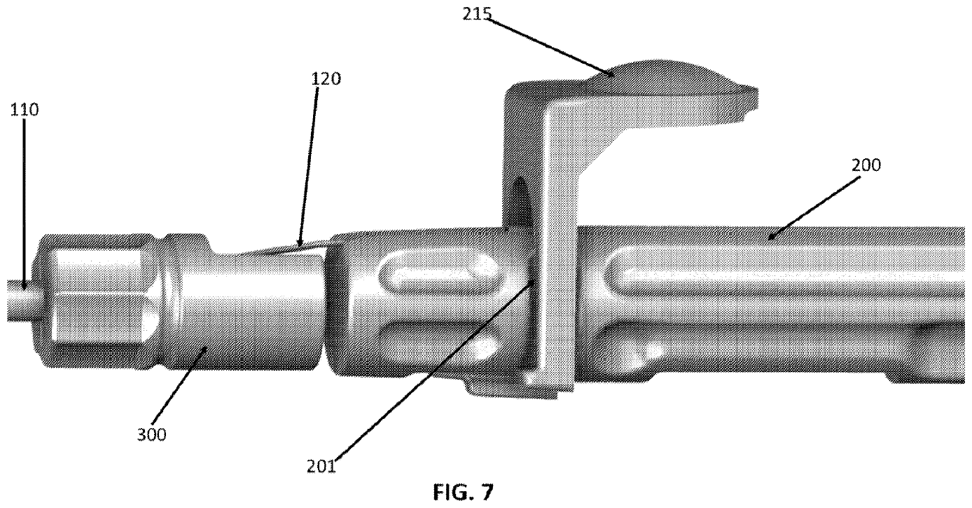

FIG. 7 provides examples of further detail of the arrangement and configuration of the valve 300, guide element 120, needle carrier 200 and actuator trigger. Here, the actuator trigger 215 is shown operably engaged to the needle carrier 200 such that the needle carrier 200, near a distal end has channels 201 or other elements configured to facilitate an engagement with the actuator trigger. In some examples, and as illustrated in FIG. 7, the actuator element may be a depressible button that can be configured to move independent from the needle carrier allowing the actuator trigger to be depressed and release the needle carrier via the actuation element (not shown).

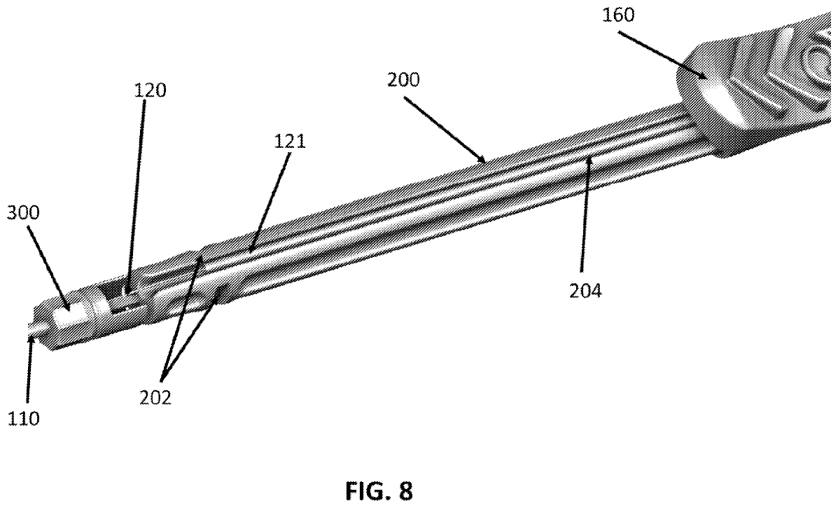

FIG. 8 shows a perspective view of IVAD components where the handle, actuation element, actuator trigger, and catheter hub have been removed or hidden from view to expose the alignment of the needle carrier 200, guide element 120, valve 300, and slider 160. In some examples, the needle carrier channel 204 may be an open channel or a closed lumen configured to route or guide the guide element through the IVAD during use. As illustrated in FIG. 8, the needle carrier channel 204 can be configured to receive the guide element 120 extending therethrough. In some examples, the guide elements may be exposed when positioned in the needle carrier channel 204. In some examples, tubing or a guide element lumen may be provided within the needle carrier channel 204 configured to facilitate the guide element as it may be advanced therethrough. With the actuator trigger removed, engagement features 202 are shown in an exemplary arrangement near the distal end of the needle carrier 200 and configured to engage with the actuator trigger (not shown) for operation of the needle carrier when the IVAD is in use. The engagement features 202 are shown here as detents or channels that can receive a portion of the actuation trigger to lock or retain the needle carrier 200 in position until the trigger is activated or depressed to release the needle carrier from the distal position in the handle to a proximal position in the handle when the needle is to be retracted.

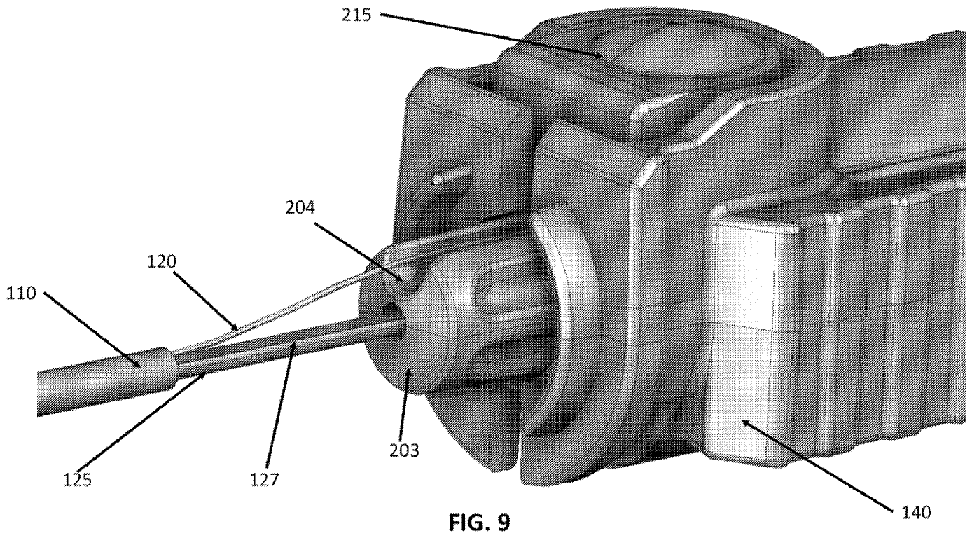

A guide element route or pathway through the IVAD may be supported by the arrangement and configuration of various components such as the needle carrier (e.g., needle carrier channel), guide element coupling on the slider, and position of the flat exterior surface of the needle. In some examples, the needle carrier distal end may be configured to facilitate a transition for the guide element from the IVAD handle distally through the catheter hub, hemostasis valve, and catheter lumen. FIG. 9 illustrates an example configuration and arrangement of the guide element transitioning from the distal end of the handle (e.g., within the handle body) out through to the catheter lumen. In this example, the valve has been removed to illustrate an example transition of the guide element 120 from the needle carrier distal end 203 and into the catheter 110 (e.g., catheter lumen). The needle 125 is shown extending generally along an axis of the needle carrier and into the catheter lumen. The guide element 120 is shown having a slight downward slope from an upper portion of the needle carrier 200 (e.g., from the channel of the needle carrier) towards the needle 125 and into the catheter 110. In some examples, as the slider advances distally, the guide element may be correspondingly advanced and a length of the guide element 120 may travel along this pass, including the sloped transition through the distal end of the handle and into the catheter 110.

The needle carrier distal end 203 may be configured with the channel 204 positioned anywhere around the perimeter. For example, the channel 204 may be positioned near a 12 o-clock position longitudinally orientated along the length of the needle carrier, as illustrated in FIG. 9. However, the channel 204 may also be positioned longitudinally long the needle carrier body as any position along the longitudinal perimeter of the needle carrier 200. In some examples, the position of the needle carrier channel 204 may be based on the position of the needle flat surface 127. For example, FIG. 9 illustrates the needle carrier channel 204 at the 12 o'clock position corresponding with FIG. 10A illustration of the needle flat surface position similarly at the 12 o'clock position. Considering this example, the guide element may be extending through the slider lumen 163 and bend downwards in the handle through the lumen 163 to accommodate the guide element routing to the 12 o'clock position of the needle carrier channel 204 and the needle flat surface positions.

FIG. 10A to FIG. 10D show cross section views of the needle, catheter, guide element as viewed from the distal end of the IVAD. In particular, FIG. 10A illustrates the needle lumen 131 generally at a central axial position with generally concentric circles including the catheter 110. The guide element 120 is shown at the top of the needle 125 and within the catheter 110. In this example, the guide element 120 has a surface corresponding to the exterior shape of the needle 125, where the guide element 120 and needle 125 contact. For example, the guide element may be advanced or retracted along the exterior surface of the needle 125 such that the guide element 120 and needle 125 contact each other along the contacting surface. FIGS. 10B, 10C and 10D illustrate examples of different arrangements and orientation of the guide element 120 about the needle 125 perimeter. The dotted lines are provided as markers to describe the positioning of the guide element within the catheter lumen. For example, the dotted lines distinguish four quadrants of each image (e.g., FIG. 10A to 10D) described clockwise from the upper left first quadrant to the lower left fourth quadrant. As an example, FIG. 10B shows the guide element positioned in a second and third quadrant, FIG. 10C show the guide element 120 in quadrants three and four; and FIG. 10D show the guide element 120 in quadrants one and three. The guide element may be locatable longitudinally along the needle surface at any position.

To accommodate the position and orientation of the guide element 120 on the needle exterior, a flat surface 127 is provided along a length of the needle body. The flat surface 127 may contact a corresponding surface of the guide element 120. Referring to the examples provided in FIG. 10A to FIG. 10D, each example of the needle 125 is shown in the same rotational position with the tissue penetrating tip 126 illustrated below the lumen 131, while the guide element 120 is illustrated at different positions around the needle exterior.

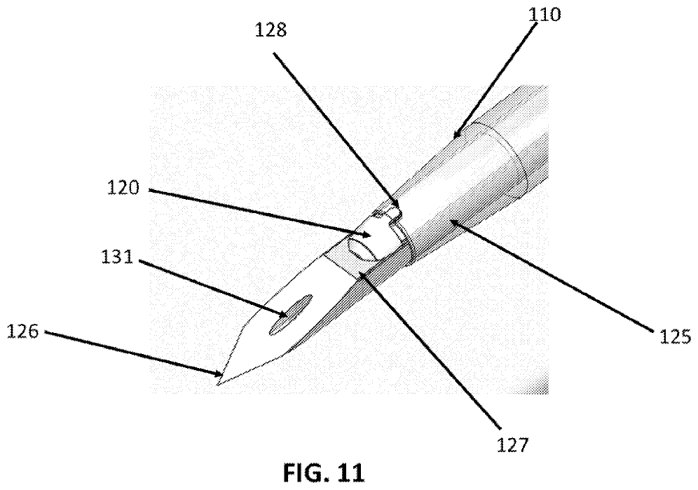

The relationship of the guide element, catheter and needle provide unprecedented adaptability in the deployment and use of the guide element. Considering current art, the guide element position is limited. However, having a needle configured to communicate with the guide element as described herein provides increased opportunity for guide element function when advancing within the vasculature. FIG. 11 shows a perspective view from a distal end of the IVAD. In particular, the tissue penetrating tip 126 of the needle 125 may indicate the distal tip of the IVAD with the guide element 120 in a retracted (e.g., ready-to-use configuration) nested into a notch 128 at the distal top of the catheter 110. In this example, the tissue penetrating tip 126, guide element 120, and distal end of the catheter 110 form a taper that can be configured to reduce the amount of physical impact during insertion (e.g., venipuncture). The guide element may be advanced and form an atraumatic tip beyond the needle tip 126. In the example shown in FIG. 11, the guide element would be advanced along the needle flat surface 127 over the needle lumen and beyond the tip 126.

FIG. 12A and FIG. 12B show detailed perspective views of a slider 160 as described herein. In some examples, a slider may comprise one or more supports to promote operation in conjunction with an IVAD. In some examples, slider supports may be a rail system 162 configured to engage the IVAD handle (e.g., the handle slot). In FIG. 12A, an example of a rail system is shown having a rail 162 (e.g., a dovetail/c-rail) locatable along a length of the slider body. A corresponding rail may be positioned on an opposite side of the slider body and together, the rails can comprise the rail system. Although not shown in FIG. 12A, it can be appreciated that the slider 160 may be positioned within the handle (e.g., slot) such that a perimeter of the slot engages the rails and retains the slider in position relative to the handle and slot. For example, the slider leg may exert some force on the slider against the slot that may include some torque by the slider on the perimeter of the slot. The rails can be configured to retain the slider in a relatively stable position within the slot while the slider is being used (e.g., traveling within the slot. The rails 162 can be configured to facilitate the stable communication of the slider 160 within the slot and reduce the amount of deviation or displacement of the slider from a consistent path when traveling within the slot. For example, the rails 162 can be configured to allow translation of the slider while preventing the slider from moving up or down given the moment created by the stopper leg when engaged with the housing.

In FIG. 12B, slider supports are shown to include slider reliefs 164. In some examples, the slider leg contacting the interior surface of the handle (e.g., ratchet system) may result in a force or moment that may cause the slider to pitch within the handle. Pitching may be undesirable during use. Accordingly, in some examples, slider reliefs may be incorporated into the slider to prevent instability and promote overall stability of the slider within the handle, before, during or after use. Slider reliefs may work in conjunction with the proximal housing. In some examples, the slider supports may be configured or keyed to the handle such that the slider is configured to have one or more supports with corresponding structural elements of the handle to promote sufficient engagement between the slider and handle to promote stability without reduced function or translation of the slider within the handle slot.

In some examples, the guide element 120 may be in communication with the slider 160. The opening or lumen 163 of the slider leg shown in FIGS. 12A and 12B may be adapted to receive the guide element (e.g., guide wire) extending therethrough. In some examples, the guide wire may be coupled to the slider 160 at the opening 163. In some examples, the guide wire may extend from the slider through the needle carrier channel 204 to the needle flat surface 127, as described herein. In some examples, the lumen 163 may have a smaller diameter distal opening as illustrated in FIG. 12A compared to the distal portion of the lumen visible in FIG. 12B. The configuration of the lumen 163 can accommodate and support clocking of the guide element path or routing from the slider through the needle carrier and to the needle flat surface. In some examples, the guide element extending through the lumen 163 may be allowed to bend through the larger diameter portion of the lumen 163 to accommodate the direction of the guide element 120 along its path through the needle carrier channel 204 and to the needle flat surface. In some examples, the needle carrier channel 204 (e.g., as illustrated in FIG. 8) may be positioned along a length of the needle carrier exterior in the same orientation as the position of the flat surface of the needle. For example, the needle carrier channel 204 may be clocked around the needle carrier body correspondingly to the clocked position of the needle flat surface, as described herein.

FIG. 12C illustrates an alternative to the position of the lumen 163 on the slider 160. In this perspective view, examples of additional or alternative openings 163a, 163b, 163c, 163d are provided to illustrate the position of the lumen may be such that it is substantially in line with the needle carrier channel 204 for routing the guide element 120 through the interior of the handle 140 to the needle flat surface 127. Guide element routing may therefore be clocked or oriented inside of the handle starting with the position of the lumen 163 (e.g., 163a, 163b, 163c, 163d) on the slider, the position of the needle carrier channel 204 on the needle carrier, and the position of the flat surface 127 on the needle exterior.

In some examples, a smaller diameter distal opening of the lumen 163 may be adapted to promote bonding of the guide element or guide element tube with the slider. For example, the smaller diameter opening may be adapted to receive the guide element extending therethrough and the distal segment of the opening may be filled with bonding to fix the guide element in position coupled with the slider.

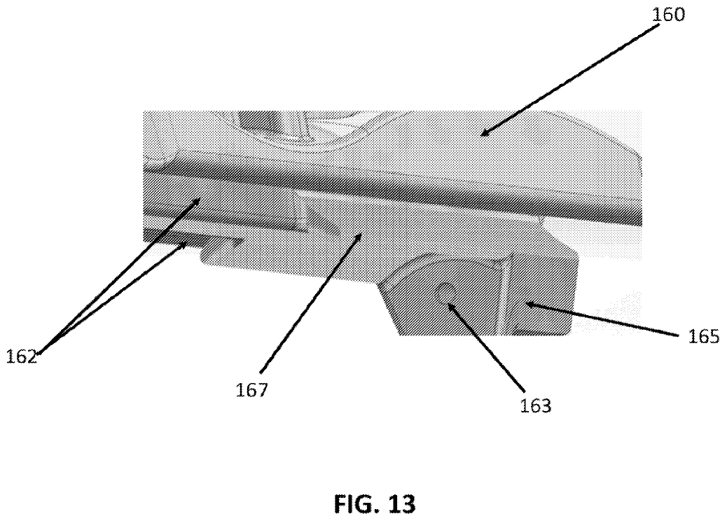

FIG. 13 shows a more detailed perspective view from underneath the slider 160. The rails illustrated herein comprises the rails 162 and a secondary set of rails that may be keyed to the handle or another component of the IVAD (e.g., needle carrier). The secondary rails may provide additional stability to support the rails 162 or may provide a stopping mechanism as a fail-safe element to prevent over rotation of the slider 160 within the handle. In some examples, the secondary rails 167 may act as a stopping mechanism to prevent the slider 160 from being displaced beyond an acceptable position. For example, the secondary rails 167 may be configured to prevent the slider 160 from advancing past a location within the handle (e.g., slot) by contacting a block or structural element such that the slider 160 is prevented from advancing beyond that point of contact.

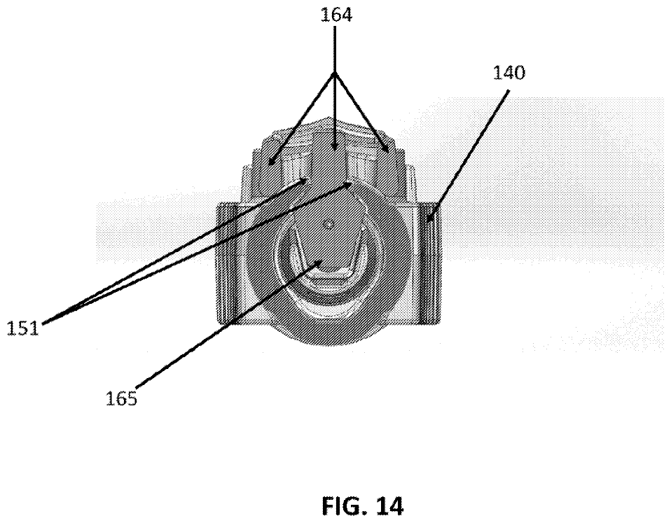

Another view of the slider 160, handle 140 and slider supports is shown in the cross-section view of FIG. 14. In particular, the slider leg 165 is shown here from the proximal end and the housing 140 forming an incomplete cylinder due to the slot included in this cross-section view. The leg is extending from the slider 160 and into the slot 150 such that the slot perimeter 151 is engaging the rail 126 of the slider 160. Slider relief 164 are shown superiorly to the handle 140 in general alignment with one another to promote stability and prevent rotational forces from inadvertently displacing the slider 160 within the slot 150.

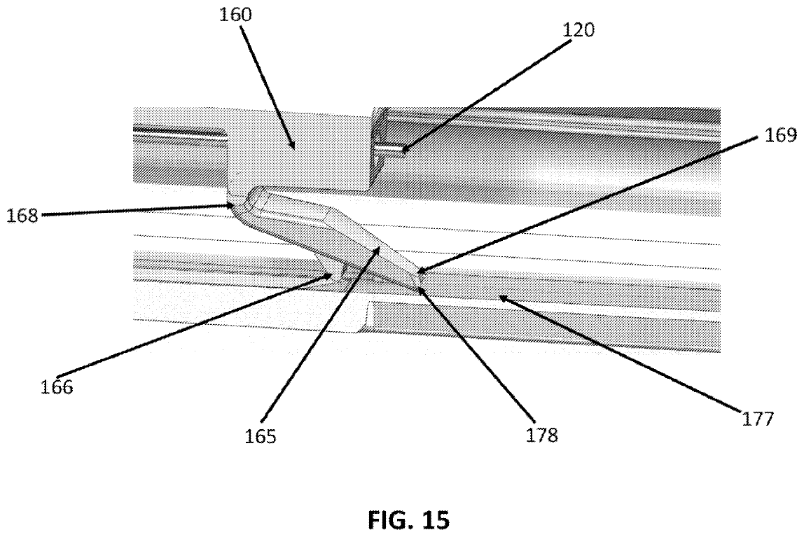

As described herein, an IVAD may arranged or describable relative to the orientation, positioning, and configuration of one or more components. For example, FIG. 15 illustrates how an IVAD may be arranged in an “armed” configuration. In some examples, the IVAD may be deliverable or in an initial armed position when the guide element tip is engaged with the catheter tip/notch 128 and the slider leg 165 is engaged to the ratchet teeth near a proximal end of the handle. Visible in FIG. 15 is a proximal portion of a ratchet features comprising a proximal stop 177 configured to prevent or inhibit further proximal translation of the slider 160. The slider leg 165 at a terminal end can contact the proximal stop 177 at a set point 178 where the leg acts to lock the slider in place to prevent any further proximal translation within the slot. Accordingly, the proximal stop 177 may also provide the user with feedback for the location of the slider within the slot (e.g., handle). For example, the proximal stop 177 may be engaged and identifiable as the slider is prevented from any further proximal translation. In some examples, the proximal stop 177 may not prevent forward translation (e.g., distal advancement) of the slider as the leg may be configured to advance distally from the proximal stop. In some examples, the proximal slot may be initially configured to have a position relative to the length of guide element required to position the guide element distal tip within the distal catheter end (e.g., catheter notch). In such an example, both the guide element and proximal stop may be configured to prevent continued proximal translation of the slider within the slot.

As described herein, a needle carrier may be displaced to withdraw a needle from within a vessel after accessing the vessel during use. For example, the needle may no longer be required for the procedure beyond the initial access and introduction of the catheter to the vessel. Accordingly, the needle carrier can be configured to translate or slide proximally within the handle thereby withdrawing the needle coupled thereto.

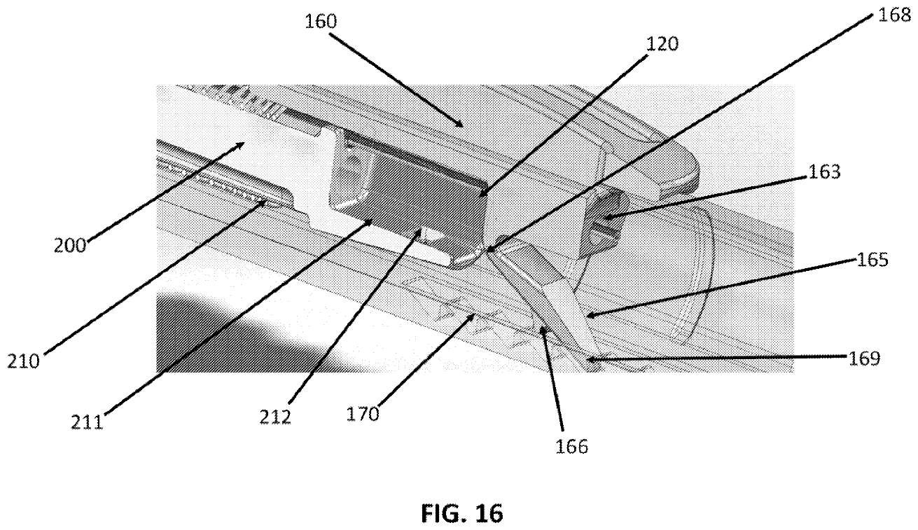

FIG. 16 to FIG. 18, detail views illustrating the engagement of the slider leg 165 and the proximal end of the needle carrier 200. In FIG. 16, the slider 165 is in a position distal to an initial position within the handle. For example, the slider may be advanced distally as a procedure may require the guide element 120 to be advanced by the slider 160.

The perspective view is also a longitudinal cross section to show the dock 211 of the needle carrier 200 at a proximal end. The dock 211 may be a concave or cylindrical depression into the needler carrier from the proximal end and configured to couple to the slider leg 165 when the slider leg 165 is sufficiently advanced into the dock 211 and the slider protrusion 166 has engaged the opening 212. The slider leg 165 is shown engaging the ratchet teeth 170 as it advances distally within the handle. The distal end of the needle carrier, as previously illustrated, may be generally retained by the handle 140 and may be prevented from moving distally, even when the slider leg 165 makes contact. The slider leg 165 has the slider foot 169 in contact with the ratchet teeth 170 as the slider knee 168 is promoting extension of the leg 165 towards the ratchet teeth. The slider knee 168 is adjacent to the needle carrier dock 211 but the slider leg has not made contact yet to bend the knee 168.

Continuing to FIG. 17, the slider leg 165 has been advanced yet further towards the needle carrier dock 211. Here, and referring to the slider knee 168, the slider leg engages the dock 211 as the slider knee 168 enters the dock 211. The knee 168 is bent as the slider 160 and slider leg 165 are advanced into the needle carrier dock 211. In this position of the sequence, the leg 165 has entered the dock, but the slider protrusion 165 has not yet entered the dock opening 212 for a positive or locked engagement between the slider and the needle carrier 200. In this view, the slider foot 169 has been raised away from the ratchet teeth and is no longer in contact with any ratchet teeth 170. Accordingly, the slider 165 may be in a position to be retracted within the housing 140.

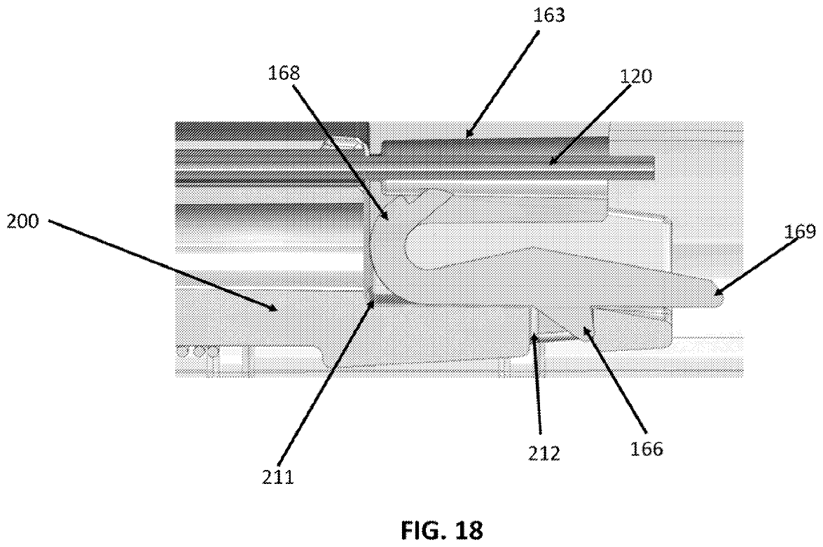

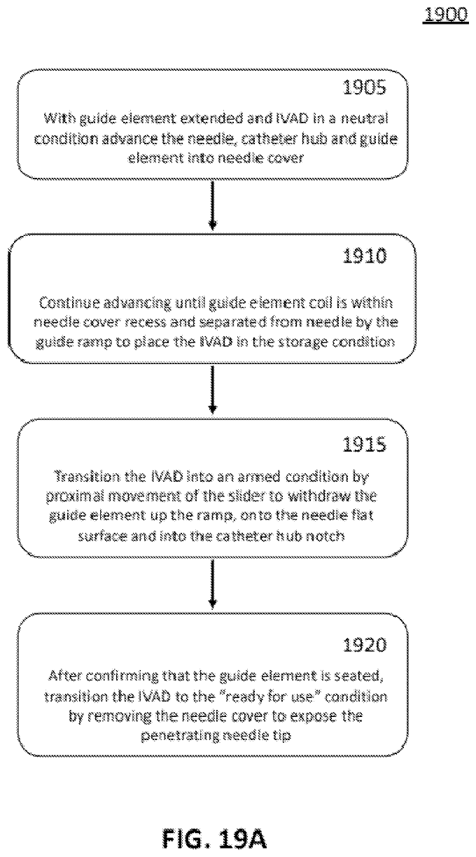

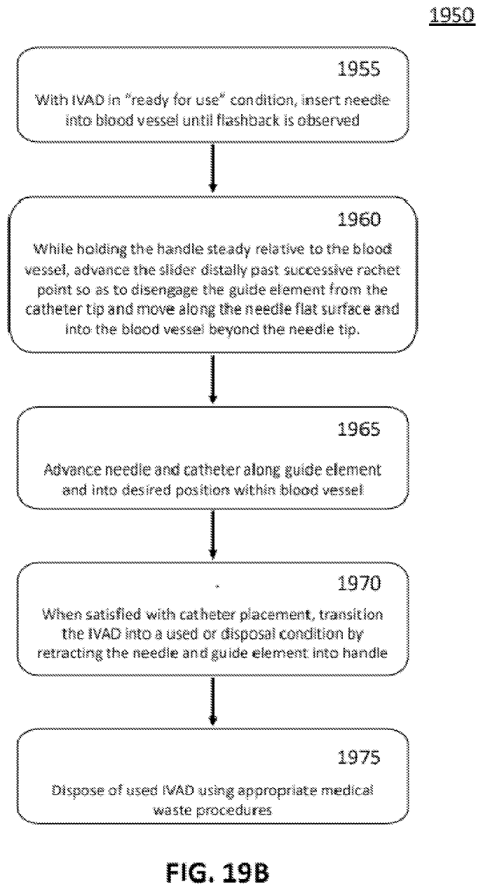

Finally, FIG. 18 illustrates an example of the slider leg 165 docked within the needle carrier 200. The slider knee 168 is at a distal location within the dock 211 and the slider leg protrusion 166 has engaged the dock opening 212 such that the slider 160 may be retained within the dock 211. In some examples, once the slider leg protrusion 166 has engaged the dock as illustrated, the slider may be prevented from further translation without correspondingly moving the needle carrier. In some examples, once the slider 160 has engaged the needle carrier dock 211, the needle carrier may be configured to retain the slider and needle carrier in place within the handle. In some examples, once the needle carrier 200 has been engaged by the slider leg 165, the slider may be retracted proximally over the ratchet teeth as the slider leg is bent or otherwise biased above or away from the ratchet teeth allowing for proximal retraction without interference from the ratchet teeth. In some examples, the slider may be used to pull or withdraw the needle and needle carrier proximally within the handle. In this way, the user may determine the amount of force, rate, and other characteristics of withdrawing the needle from the vessel as they manually retract the needle carrier into the handle. In some examples, the actuation element may be triggered or triggerable by the engagement of the slider leg 165 engaging with the proximal end of the needle carrier. For example, the slider leg may act as the actuator trigger to trigger the displacement of the needle carrier 200 by the actuation element when the slider leg 165 engages the needle carrier dock 211.

In some examples, the slider leg 165 and the plurality of teeth 170 on the inner wall are adapted and configured to act like a ratchet system to ensure only one way and irreversible movement of the slider into a more distal position. This rachet system is engaged until the distal advancement of the slider 160 seats the slider leg 165 into the is nested into the needle carrier dock 211. When the slider leg is fully seated in the needle carrier dock 211, the slider protrusion 166 seats within the dock opening 212 and mechanically locks the slider to the carrier dock. At this point, with the slider leg withdrawn, the slider may be advanced proximally and distally freely since the needle carrier is above and clear of the rachet teeth 175. In some examples, it is after the slider 160 has docked to the needle carrier 200, in this manner, that the actuation button may be depressed to proximally retract the needle carrier-slider combination proximally within the handle, thereby withdrawing the needle and guide element leaving the catheter in place in the vessel.

In use, an IVAD as described herein may increase the safety and efficacy of vascular access procedures. The integrated guide element and associated deployment systems can increase the success of first pass placement of a catheter within a vessel due to the easy and intuitive use of the slider to manipulate the guide element and the ratcheting system for real-time feedback for the user to determine the position, placement and other characteristics of the guide element during use.

In general, an IVAD as described herein may be used by inserting the needle (e.g., tissue penetrating tip) into a patient's vessel. The guide element and distal catheter tip may act as a dilator to introduce the catheter into the vessel corresponding to the entry created by the needle. In some examples, the guide element and distal catheter tip may have a tapered geometry to gently expand the opening into the vessel sufficient for the catheter without unnecessary insult to the patient's tissues. The user may then engage the slider and distally advance the slider within the slot such that the slider correspondingly advances the guide element along the needle and distally from the catheter tip. The guide element may be configured to form an atraumatic distal end after passing the needle tip to prevent further injury once inside the vessel. Once the user has sufficiently advanced the guide element into the tip, the catheter may be placed, and the needle can be withdrawn in a simplified and concise manner.

It is to be appreciated that the various different IVAD embodiments and configurations may undergo different forms of preparation and use. By way of example, a user may prepare the IVAD by establishing the configuration of the IVAD. Different configurations of the IVAD may include the armed configuration, ready-to-use configuration, neutral configuration, stored configuration, etc. In any configuration, the user may orientate the guide element distal tip, needle distal tip, catheter distal tip, slider, handle, needle carrier, etc. relative to one another such that the IVAD is prepared in a configuration for accessing a blood vessel. Then, the user inserts the needle into a vessel of the patient. In some examples, the catheter hub may indicate sufficient vascular penetration with a flash or blood or other liquid visible within the catheter hub. After the needle has gained access to the vessel, the guide element distal tip and catheter distal tip may also have gained access to the vessel as the needle is generally positioned within the catheter lumen and the guide element is generally positioned between the needle exterior and catheter lumen interior. The user may then advance the slider distally within the handle slot. When advancing the slider, the user may apply pressure to the slider as it is advanced within the slot to sufficiently engage a length of the guide element such that the guide element is correspondingly advanced with the slider.

In some embodiments, as the slider is advanced the slider leg contacts the ratchet teeth and may provide feedback including an indication that is audible, tactile, visual or combination thereof. For example, the user may advance the slider and feel the clicks of the slider leg sliding over the ratchet teeth.

In alternative methods of use, a user may determine that the guide element is sufficiently placed or advanced within the vessel and may then retract the needle from within the vessel. The user may trigger the actuation element by engaging the actuator trigger to release the needle carrier allowing the activation element to bias the needle carrier and needle proximally within the handle. In some examples, the slider leg may be advanced to engage the needle carrier dock where the slider may then be retracted within the handle to manually retract or withdraw the needle into the handle. The catheter hub may then be disengage or uncoupled from the handle and used in accordance with any other medical device or continuation of a medical procedure utilizing the access to the vessel.

FIG. 19A is a flow chart of an exemplary method 1900 of a process of placing a neutral condition IVAD into a needle cover and transitioning a neutral condition IVAD into an armed condition and a ready for use condition. First, at step 1905, with the guide element extended and the IVAD in a neutral condition, advancing the needle, catheter hub and guide element into the needle cover 400.

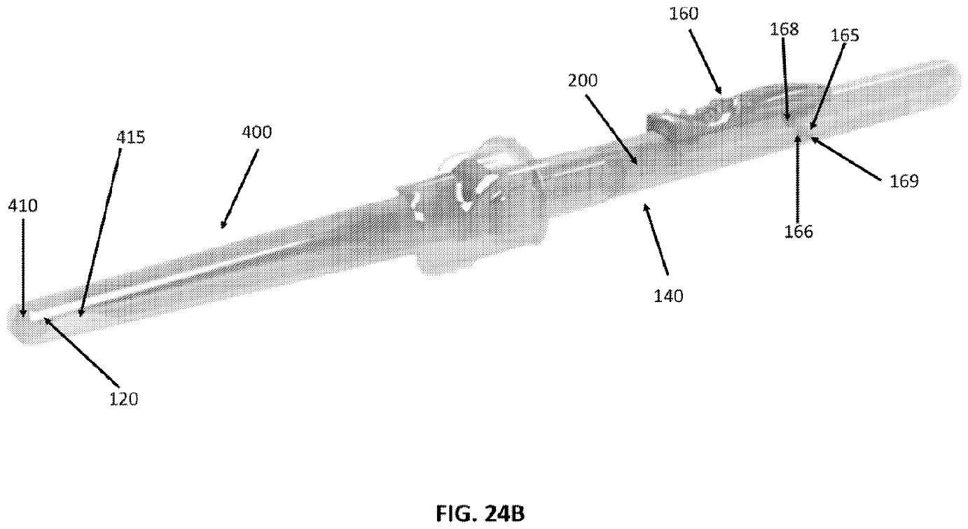

Next, at step 1910, there is a step of continuing to advance the IVAD until the guide element coil is within the needle cover recess 410. In the recess, the end of the guide element is separated from the needle by the ramp 415. With the guide element in the recess and the needle cover secured to the handle as in FIG. 24A and FIG. 24B, the IVAD is in a stowed or neutral condition. This is a good condition for long term storage of an IVAD since the coiled, curved or shaped features of the distal most end of the guide element are maintained within the recess.

Next, when the user wants to use the IVAD, the IVAD is transitioned into an armed condition by proximal movement of the slider to withdraw the guide element up the ramp, onto the needle flat surface and into the catheter notch. (step 1915).

Next, at step 1920, after confirming that the guide element is seated, the slider is in the correct position or otherwise confirming that the IVAD is armed, the IVAD is transitioned from armed to ready for use by separating the needle cover from the handle to expose the penetrating needle tip.

FIG. 19B is a flow chart of an exemplary method 1950 of a process using an IVAD in a ready for use condition to enter into a blood vessel. At step 1955, with the IVAD in a ready to use condition, insert the needle into the blood vessel until flashback is observed.

Next, at step 1960, while holding the handle steady relative to the blood vessel, advance the slider distally past successive rachet points so as to disengage the guide element from the catheter tip and move along the needle flat surface and into the blood vessel beyond the needle tip.

Next, at step 1965, advance the needle and catheter hub along the guide element into a desired position within the blood vessel.

Next, at step 1970, when the user is satisfied with the catheter placement, the IVAD is transitioned into a used or disposal condition by retracting the needle and guide element into the handle. Thereafter, dispose of the used IVAD using appropriate medical waste procedures. (step 1975).

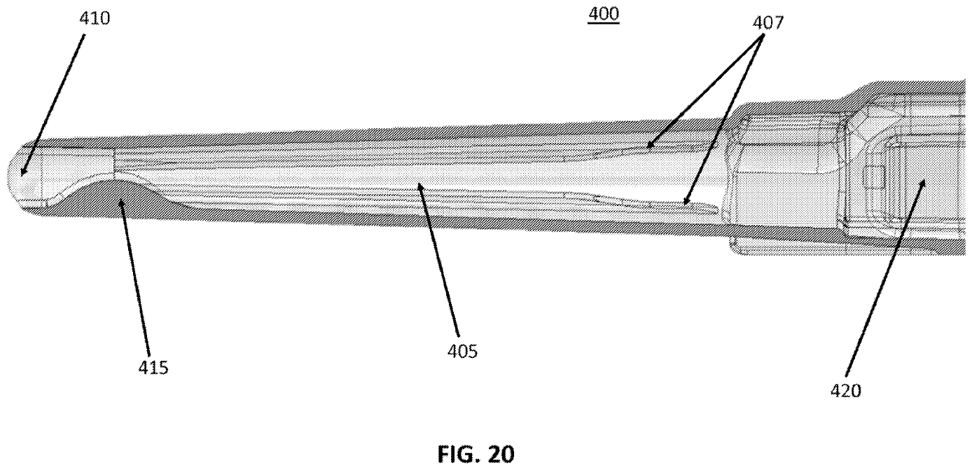

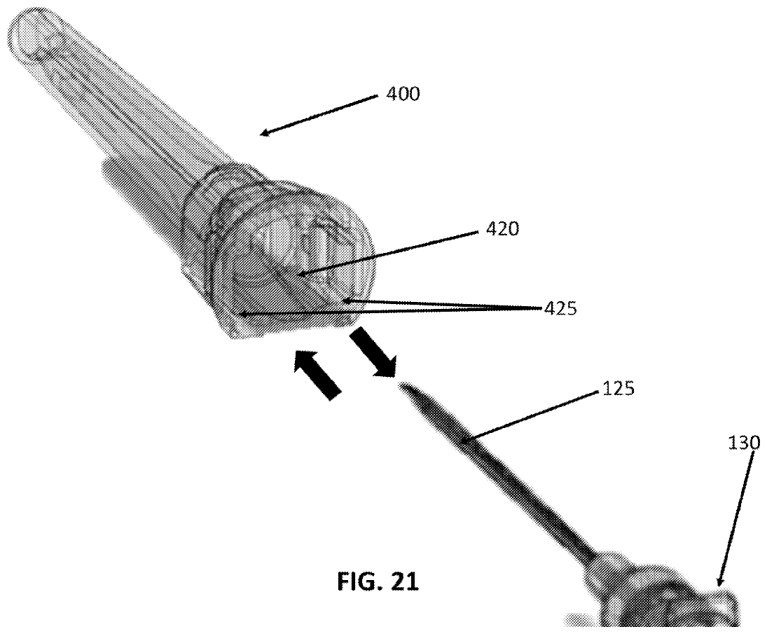

FIG. 20 is a cross section view of a needle cover 400. The needle cover 400 has a proximal opening 420 and lumen 405 adapted and configured to receive the needle, guide element and catheter hub. Additionally, the opening 420 may be sized for a friction fit or have corresponding mating elements to releasably attach to the handle. FIG. 21 is a rear isometric view of the needle cover of FIG. 20 adjacent to a piercing needle tip at the distal end of the needle 125.

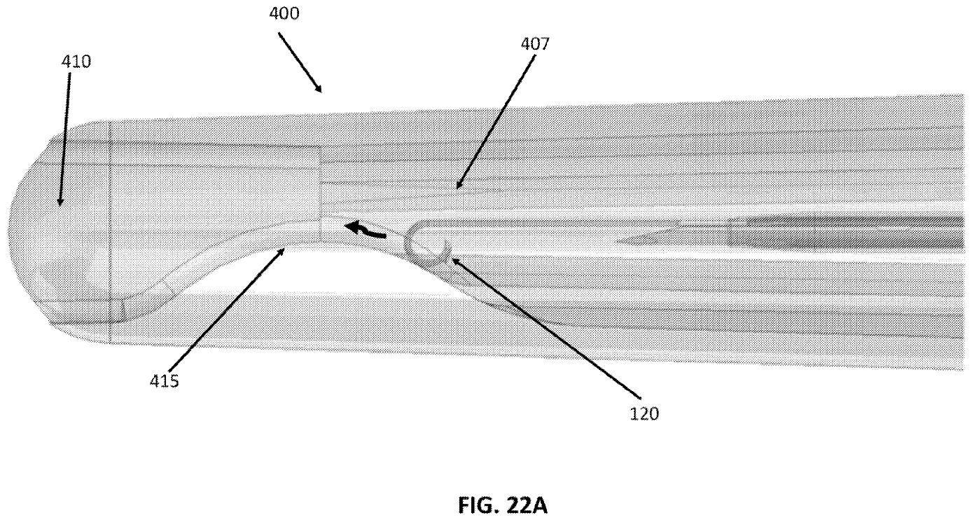

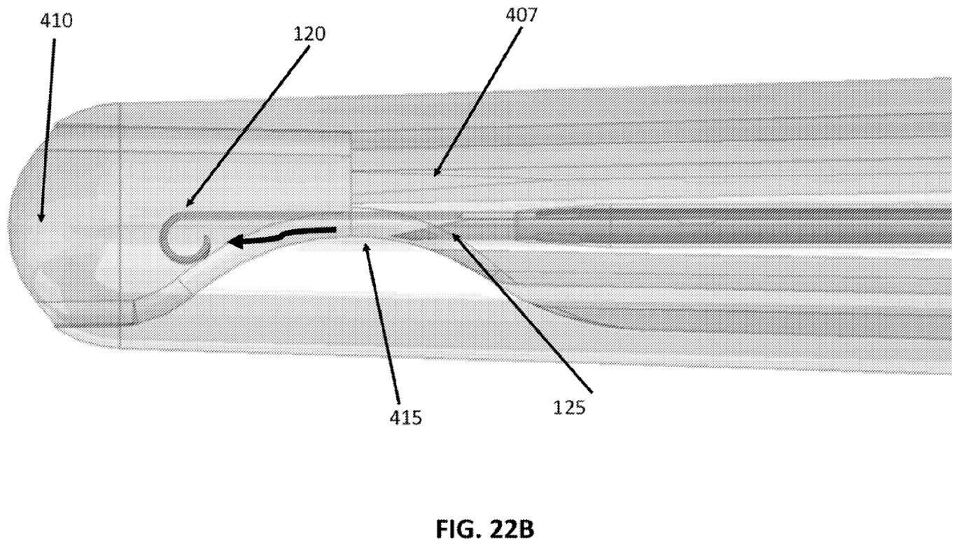

The lumen 405 includes features such as the ramp 415 and a recess 410. The ramp 415 is used to guide and protect the guide element from possible damage by contact with the piercing needle tip. The shape of ramp 415 interacts with the guide element as better appreciated by reference to FIG. 22A and FIG. 22B. Also seen in the cap 400 from this view are alignment features extending along an interior of the lumen 405. The alignment features 407 can be configured to align the distal end of the IVAD when the cap is put on or removed from the distal end of the IVAD.

FIG. 22A and FIG. 22B are enlarged cross section views of a guide element advancing up a ramp 415 (FIG. 22A) and then down the ramp 415 (FIG. 22B) that separates the guide element from the needle tip and guides the guide element into a recess 410 in the needle cover as shown in FIG. 22B. In some examples, the transition of the guide element over the ramp 415 can provide for a loading or transition of the IVAD to an armed configuration with the guide element 120 drawn back into position over the needle flat surface and seated into the notched distal end of the catheter. The tissue penetrating tip 126 of the needle 125 may be restricted from advancing into the ramp 415 to prevent contact between the tissue penetrating tip 126 and the ramp 415.

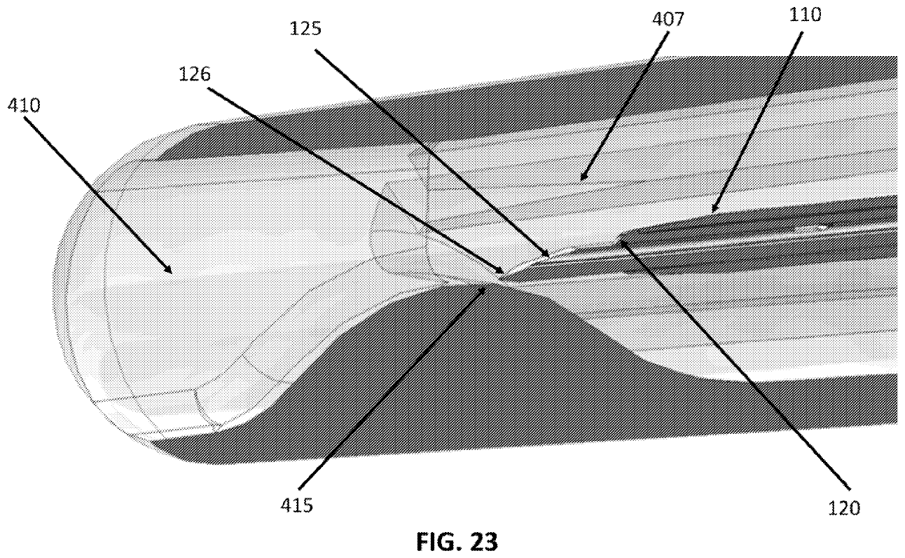

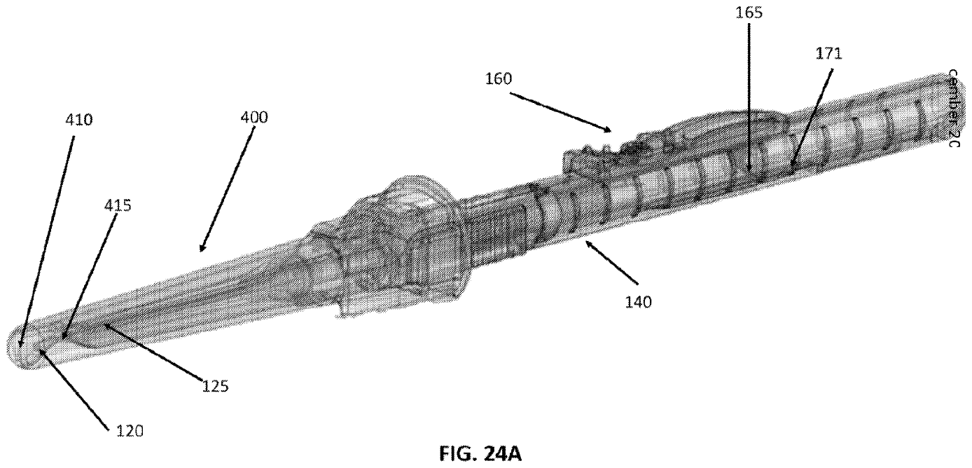

FIG. 23 is an enlarged cross section of the IVAD of FIG. 22B showing the relative positions of the piercing needle tip to the ramp. The guide element is removed for clarity. In use, the needle tip does not contact the interior of the needle cover, the ramp 415 or the guide element.

FIG. 24A and FIG. 24B are isometric detailed interior and isometric transparent views, respectively, of the IVAD as configured in a storage condition of FIG. 22B. This is the condition of the IVAD at the conclusion of the guide element movement shown in FIG. 22A and FIG. 22B. The cap 400 is coupled with the distal end of the handle 140 to prevent contact or damage to the catheter hub 130, needle 125, guide element 120, or other features extending beyond the distal end of the handle 140.

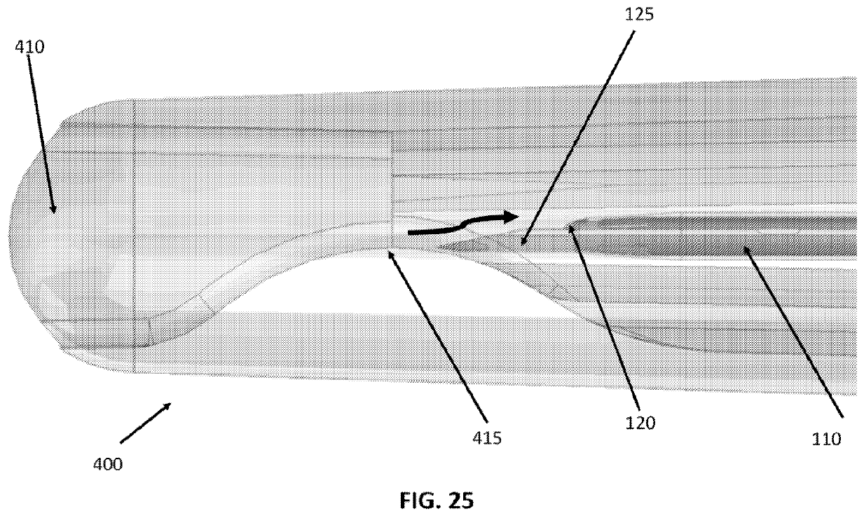

FIG. 25 is a cross section view showing the movement of the guide element along the ramp and into position against the catheter tip during the transition into an armed condition.

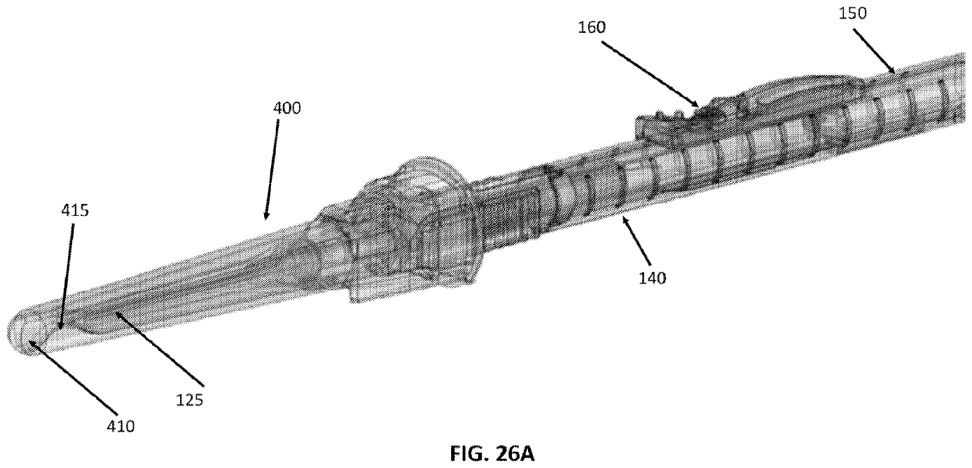

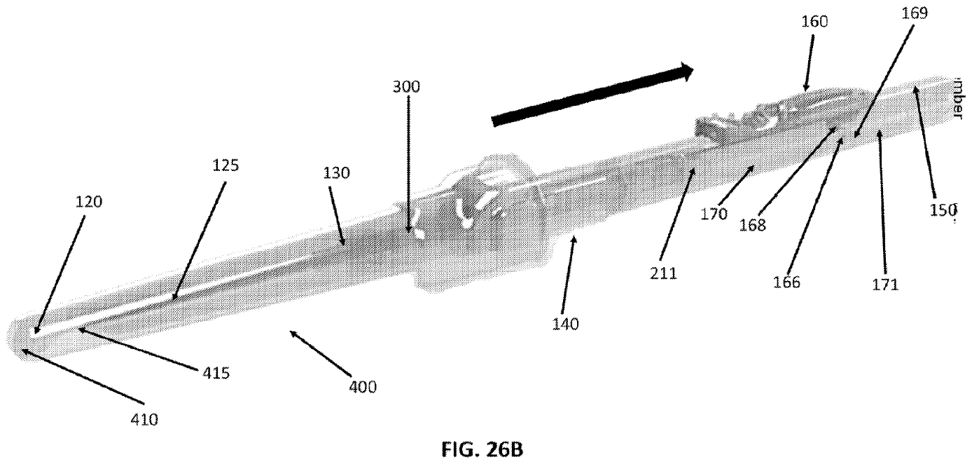

FIGS. 26A and 26B are isometric detailed interior and isometric transparent views of an IVAD in an armed condition where the guide element has been transitioned into position as shown in FIGS. 24 and 25. FIG. 26B illustrates the direction of the slider to accomplish the guide element movement shown in FIG. 25.

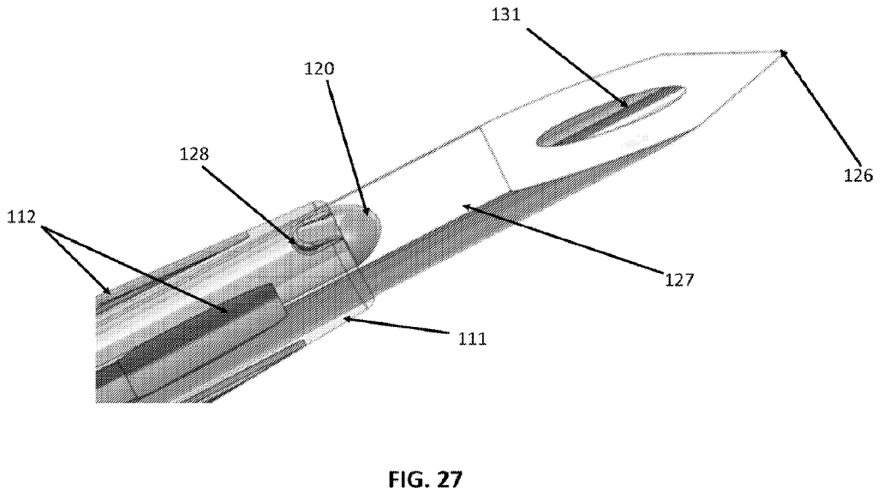

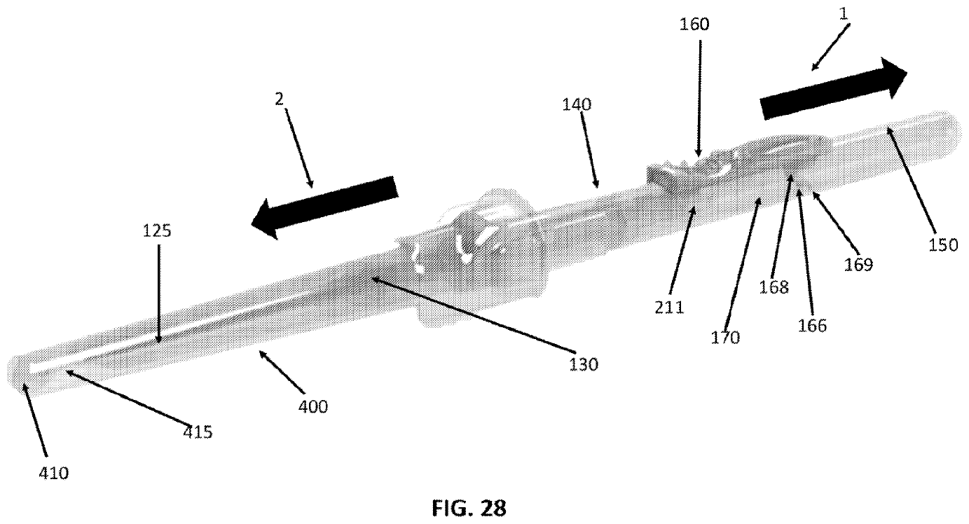

FIG. 27 is a perspective view of the distal end of an armed IVAD as shown in FIG. 26A and FIG. 26B with the needle cover removed as shown in FIG. 21 to transition the IVAD into a ready to use condition.

FIG. 28 is an isometric view of an IVAD in a neutral condition as in FIG. 24A and FIG. 24B having a pictogram affixed to the needle cover and the slider indicating the order of movements to transition the IVAD from a neutral or storage condition to a ready to use condition. The pictogram indicates that the first movement is of the slider in a proximal direction as illustrated by the arrow 1. The second movement of separating the needle cover from the handle is illustrated by the arrow 2.

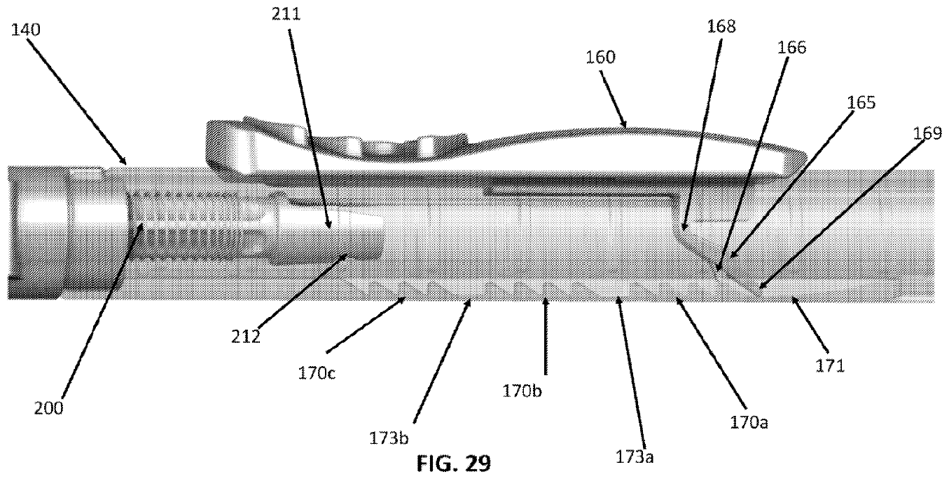

In some examples, ratchet systems (e.g., teeth 170, trough 171, proximal stop 177, etc.) and other features of the IVAD may be adapted to provide indication of deployment length of the guide element. When the slider is advanced distally, after the needle has entered a vessel, the position of the guide element distal end may be identified by the length of slider travel through the slot of the handle. FIG. 29 illustrates an example of an alternative ratchet teeth configuration having a proximal space 173a and a distal space 173b in the line or row of the plurality of ratchet teeth 170. In this example, the ratchet teeth may be described relative to their proximal or distal position in the handle. For example, a first set of proximal ratchet teeth 170a may be positioned proximal to the space 173a. A medial segment of ratchet teeth 170b and a distal segment of ratchet teeth 170c may indicate the extent of deployment of the guide element based on the slider leg engagement with the corresponding segment of ratchet teeth.

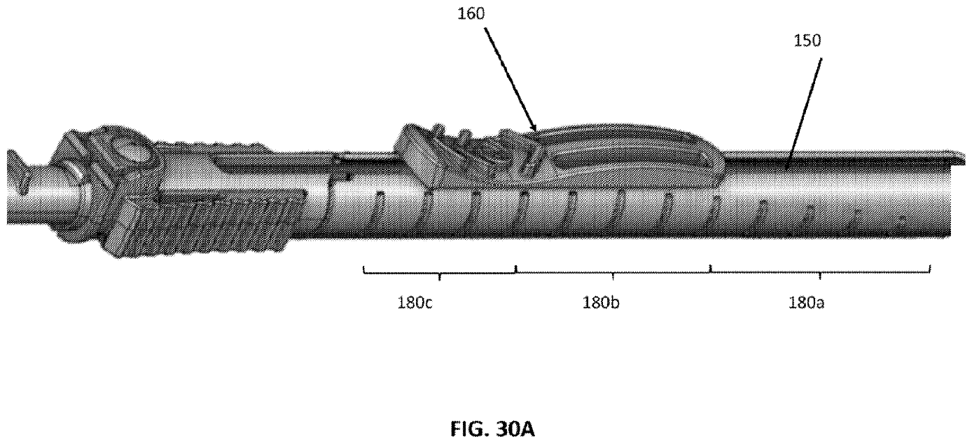

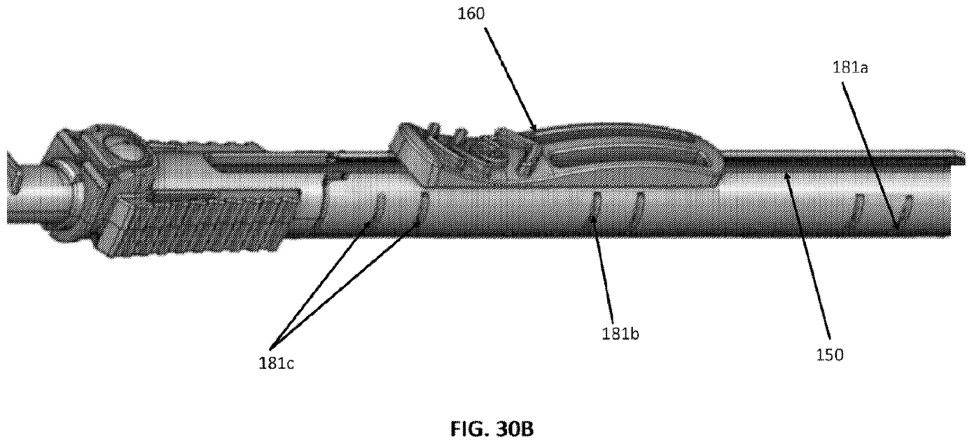

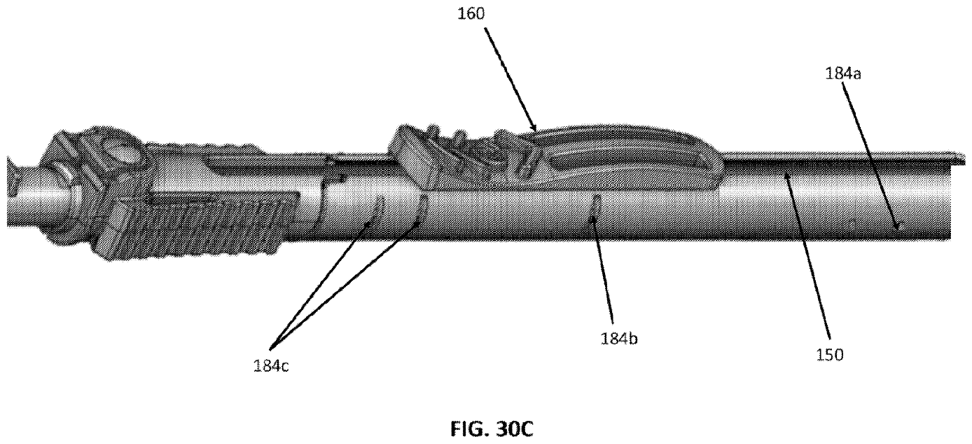

FIGS. 30A, 30B and 30C are prospective views of a handle of a vascular access device handle with features on an exterior surface and along the length of the slot. The features can be tactile, visual of both. The segments can correspond to those of the rachet as described herein such as having intermittent spacing (FIG. 29) or constant spacing (FIG. 5B) or the proximal ratchet trough (FIG. 5B) that correspond to the status of the vascular access device or the relative position of components of the access device. For example, the markings may be used to indicate if the distal end of the guide element is distal to the distal end of the needle (as when in a needle cover in FIG. 22B), seated in the notched end of the catheter (FIG. 11, 23, or 27) or beyond the distal most end of the needle (see, e.g., FIGS. 1 and 2). As a result, the position or placement of the guide element tip relative to the needle or the catheter can be included in the provided feedback for the slider position. Advantageously, this allows for the slider position to be used as an indicator of guide element position relative to the needle tip as well as progress in advancing the guide element to final deployment length. For example, as best seen in FIG. 30A, there may be a segment with a series of evenly spaced markers. The markers may be grouped as proximal deployment markers 180a, medial deployment markers 180b and distal markers 180c. In one variation, some of the markers are only along the bottom of the handle as in segment 180a. This differs from the markers in segments 180b, 180c that nearly completely encircle the handle. In contrast to FIG. 30A, FIG. 30B provides a pair of markers at distinct points on each of the segments. In another variation, FIG. 30C provides point markers 184a in the proximal end, a single band marker 184b at the middle segment and a pair of markers to denote the distal segment 184c. The placement and type of markers used and their placement along the handle may be used to provide a tactile or visual indication to the user about whether the access device is loaded, in deployment or in a fully extended guide element configuration among others.

Additional aspects of the construction and operation of a catheter placement device which includes a housing or handle having mechanism for advancing a guide structure or guide element which carries the catheter where the handle is adapted to automatically retract both the access needle and the guide structure or guide element from the catheter after the placement procedure is complete, a button activated automatic needle and guide withdrawal assembly are described in U.S. Patent Publication US 2008/0300574 and U.S. Pat. No. 9,522,254, each of which is incorporated herein by reference in their entirety.

Still other details of representative intravascular catheter insertion devices and methods are described in U.S. Pat. Nos. 5,704,914 and 5,800,395 and in U.S. Patent Publications US 2010/0094310; US 2010/0210934; and US 2012/0197200, the full disclosure of each of these are incorporated herein by reference in their entirety.

There may be one or more variations, alternatives, constructions, compositions, and/or components described herein that can be used to modify an element, component, device, system, process, etc. of a guide element, intravascular access device and/or an associated structure or process. Accordingly, any variation, description, example, element, component, process, method, method step, etc. described herein can be used as a modification, variation, and/or alternative to any element, device, system, composition, example, component, process, method, method step, etc. described in co-pending U.S. Provisional Patent Application No. 63/328,732, filed on Apr. 7, 2022, entitled “INTRAVASCULAR CATHETER WITH INTEGRATED GUIDE STRUCTURE”; and/or PCT application number PCT/US2021/54046, filed on Oct. 7, 2021, entitled “INTRAVASCULAR CATHETER WITH INTEGRATED GUIDE STRUCTURE” the entireties of which are incorporated herein.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

Throughout this specification and the claims which follow, unless the context requires otherwise, the word “comprise”, and variations such as “comprises” and “comprising” means various components can be co-jointly employed in the methods and articles (e.g., compositions and apparatuses including device and methods). For example, the term “comprising” will be understood to imply the inclusion of any stated elements or steps but not the exclusion of any other elements or steps.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about or approximately that value, unless the context indicates otherwise. For example, if the value “10” is disclosed, then “about 10” is also disclosed. Any numerical range recited herein is intended to include all sub-ranges subsumed therein. It is also understood that when a value is disclosed that “less than or equal to” the value, “greater than or equal to the value” and possible ranges between values are also disclosed, as appropriately understood by the skilled artisan. For example, if the value “X” is disclosed the “less than or equal to X” as well as “greater than or equal to X” (e.g., where X is a numerical value) is also disclosed. It is also understood that the throughout the application, data is provided in a number of different formats, and that this data represents endpoints and starting points, and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Although various illustrative embodiments are described above, any of a number of changes may be made to various embodiments without departing from the scope of the invention as described by the claims. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the invention as it is set forth in the claims.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

What is claimed is:

1. An intravascular access device, comprising:
a handle having a proximal end and a distal end, the distal end adapted to be releasably coupled to a proximal end of an intravascular catheter;
a slot in a surface of the handle, the slot having a distal end and a proximal end;
a plurality of ratchets along an interior wall of the handle;
a slider extending through and configured to translate along the slot;
a slider leg extending from the slider into an interior portion of the handle, the slider leg having a slider foot configured to engage and advance along the plurality of ratchets as the slider advances along the slot;
a needle carrier having a distal end and a proximal end, the needle carrier disposed within the interior portion of the handle, the needle carrier distal end is coupled to an access needle and the needle carrier proximal end has a carrier dock sized and configured to receive a portion of the slider; and
a guide element coupled to a portion of the slider within the interior portion of the handle, the guide element extending along the needle carrier, exiting the interior portion of the handle and extending beyond the distal end of the handle.

2. The intravascular device of claim 1, further comprising a slider protrusion on the slider leg adapted and configured to engage with a dock opening in the carrier dock.

3. The intravascular device of claim 1, wherein when the slider is in the distal end of the slot a slider protrusion on the slider leg is engaged with a dock opening in the carrier dock.

4. The intravascular device of claim 1, further comprising a slider knee on the slider leg configured to flex in response to the movement of the slider foot along the plurality of ratchets.

5. The intravascular device of claim 1, further comprising a proximal ratchet trough within the interior of the handle and proximal of the most proximal ratchet of the plurality of ratchets.

6. The intravascular device of claim 5, wherein when the slider is in a proximal end portion of the slot, the slider foot is in the proximal ratchet trough.

7. The intravascular device of any of claims 1-6, wherein the plurality of ratchets are evenly spaced along the interior wall of the handle.

8. The intravascular device of any of claims 1-6, wherein a proximal space and a distal space are within the plurality of ratchets dividing the plurality of ratchets into a proximal set of ratchet teeth, a medial set of ratchet teeth and a distal set of ratchet teeth.

9. The intravascular device of any of claim 8, further comprising visual markings or tactile markings or combination markings on the handle surface corresponding to any of the position of the slider relative to a proximal space, a distal space are within the plurality of ratchets, a proximal set of ratchet teeth, a medial set of ratchet teeth and a distal set of ratchet teeth.

10. The intravascular device of claim 8, wherein when the slider is positioned to place the slider foot in the proximal ratchet trough the guide element is adjacent to the needle and a guide element tip is seated in a notched catheter tip.

11. The intravascular device of claim 8, wherein when the slider is positioned to place the slider foot in the proximal space the guide element tip is spaced apart from the notched catheter tip.

12. The intravascular device of claim 8, wherein when the slider is positioned to place the slider foot in the distal space the guide element tip is spaced apart from a distal most end of the needle.

13. An intravascular access device, comprising:
a handle having a proximal end and a distal end, the distal end adapted to be releasably coupled to a proximal end of an intravascular catheter;
a slot in a surface of the handle, the slot having a distal end and a proximal end;
a slider extending through and configured to translate along the slot;
a slider leg extending from the slider into an interior portion of the handle,
a needle carrier having a distal end and a proximal end, the needle carrier disposed within the interior portion of the handle, the needle carrier distal end is coupled to an access needle and the needle carrier proximal end has a carrier dock sized and configured to receive a portion of the slider; and
a guide element coupled to a portion of the slider within the interior portion of the handle, the guide element extending along a channel the needle carrier, exiting the interior portion of the handle and extending beyond the distal end of the handle, the guide element comprising a flat side that corresponds with and is positioned alongside a flat side of the access needle, wherein the guide element is coupled to a portion of the slider that aligns directly with the location of the flat side of the needle.

14. The intravascular access device of claim 13, further comprising a plurality of ratchets along an interior wall of the handle and a slider foot on the slider leg configured to engage and advance along the plurality of ratchets as the slider advances along the slot.

15. The intravascular device of claim 14, further comprising a slider protrusion on the slider leg adapted and configured to engage with a dock opening in the carrier dock.

16. The intravascular device of claim 14, wherein when the slider is in the distal end of the slot a slider protrusion on the slider leg is engaged with a dock opening in the carrier dock.

17. The intravascular device of claim 14, further comprising a slider knee on the slider leg configured to flex in response to the movement of the slider foot along the plurality of ratchets.

18. The intravascular device of claim 14, further comprising a proximal ratchet trough within the interior of the handle and proximal of the most proximal ratchet of the plurality of ratchets.

19. The intravascular device of claim 18, wherein when the slider is in a proximal end portion of the slot, the slider foot is in the proximal ratchet trough.

20. The intravascular device of any of claims 15-19, wherein the plurality of ratchets are evenly spaced along the interior wall of the handle.

21. The intravascular device of any of claims 15-19, wherein a proximal space and a distal space are within the plurality of ratchets dividing the plurality of ratchets into a proximal set of ratchet teeth, a medial set of ratchet teeth and a distal set of ratchet teeth.

22. The intravascular device of any of claim 21, further comprising visual markings or tactile markings or combination markings on the handle surface corresponding to any of the position of the slider relative to a proximal space, a distal space are within the plurality of ratchets, a proximal set of ratchet teeth, a medial set of ratchet teeth and a distal set of ratchet teeth.

23. The intravascular device of claim 21, wherein when the slider is positioned to place the slider foot in the proximal ratchet trough the guide element is adjacent to the needle and a guide element tip is seated in a notched catheter tip.

24. The intravascular device of claim 21, wherein when the slider is positioned to place the slider foot in the proximal space the guide element tip is spaced apart from the notched catheter tip.

25. The intravascular device of claim 21, wherein when the slider is positioned to place the slider foot in the distal space the guide element tip is spaced apart from a distal most end of the needle.

26. An intravascular access device, comprising:
a handle having a proximal end and a distal end, the distal end adapted to be releasably coupled to a proximal end of an intravascular catheter;
a slot in a surface of the handle, the slot having a distal end and a proximal end;
a slider extending through and configured to translate along the slot;
a slider leg extending from the slider into an interior portion of the handle,
a needle carrier having a distal end and a proximal end, the needle carrier disposed within the interior portion of the handle, the needle carrier distal end is coupled to an access needle and the needle carrier proximal end has a carrier dock sized and configured to receive a portion of the slider; and
a guide element coupled to a portion of the slider within the interior portion of the handle, the guide element extending along a channel the needle carrier, exiting the interior portion of the handle and extending beyond the distal end of the handle, the guide element comprising a flat side that corresponds with and is positioned alongside a flat side of the access needle, wherein the guide element comprises a transition segment from a top portion of the slider to the location of the flat side of the needle.

27. The intravascular access device of claim 26, wherein the guide element transition segment couples the slider to a needle flat side located on a right side portion of the handle, a needle flat side located on a left side portion of the handle, or a needle flat side located on a bottom portion of the handle.

28. The intravascular access device of claim 26, further comprising a plurality of ratchets along an interior wall of the handle and a slider foot on the slider leg configured to engage and advance along the plurality of ratchets as the slider advances along the slot.

29. The intravascular device of claim 26, further comprising a slider protrusion on the slider leg adapted and configured to engage with a dock opening in the carrier dock.

30. The intravascular device of claim 26, wherein when the slider is in the distal end of the slot a slider protrusion on the slider leg is engaged with a dock opening in the carrier dock.

31. The intravascular device of claim 26, further comprising a slider knee on the slider leg configured to flex in response to the movement of the slider foot along the plurality of ratchets.

32. The intravascular device of claim 26, further comprising a proximal ratchet trough within the interior of the handle and proximal of the most proximal ratchet of the plurality of ratchets.

33. The intravascular device of claim 26, wherein when the slider is in a proximal end portion of the slot, the slider foot is in the proximal ratchet trough.

34. The intravascular device of any of claims 28-33, wherein the plurality of ratchets are evenly spaced along the interior wall of the handle.

35. The intravascular device of any of claims 28-33, wherein a proximal space and a distal space are within the plurality of ratchets dividing the plurality of ratchets into a proximal set of ratchet teeth, a medial set of ratchet teeth and a distal set of ratchet teeth.

36. The intravascular device of any of claim 35, further comprising visual markings or tactile markings or combination markings on the handle surface corresponding to any of the position of the slider relative to a proximal space, a distal space are within the plurality of ratchets, a proximal set of ratchet teeth, a medial set of ratchet teeth and a distal set of ratchet teeth.

37. The intravascular device of claim 35, wherein when the slider is positioned to place the slider foot in the proximal ratchet trough the guide element is adjacent to the needle and a guide element tip is seated in a notched catheter tip.

38. The intravascular device of claim 35 wherein when the slider is positioned to place the slider foot in the proximal space the guide element tip is spaced apart from the notched catheter tip.

39. The intravascular device of claim 35, wherein when the slider is positioned to place the slider foot in the distal space the guide element tip is spaced apart from a distal most end of the needle.

40. The intravascular device of any of the above claims further comprising:
an intravascular catheter having a lumen configured to accommodate the access needle extending therethrough and a notched distal end portion sized and configured to receive the guide element tip.

41. A cover for a vascular access device comprising:
A body having a closed distal end and an open proximal end defining an interior portion, the open proximal end sized and configured to releasably couple to a distal end of a handle of the vascular access device;
A pair of alignment features within the interior portion defining a lumen extending from the open proximal end to a recess in a distal most portion of the interior portion; and
A ramp along the interior portion proximal to recess.

42. The cover of claim 41, further comprising an intravascular access device coupled to the open proximal end of the cover.

43. The cover of claim 42, the intravascular access device further comprising:
a handle having a proximal end and a distal end, the distal end adapted to be releasably coupled to a proximal end of an intravascular catheter;
a slot in a surface of the handle, the slot having a distal end and a proximal end;
a slider extending through and configured to translate along the slot;
a needle carrier having a distal end and a proximal end, the needle carrier disposed within the interior portion of the handle, the needle carrier distal end is coupled to an access needle and the needle carrier proximal end has a carrier dock sized and configured to receive a portion of the slider; and
a guide element coupled to a portion of the slider within the interior portion of the handle, the guide element extending along the needle carrier, exiting the interior portion of the handle and extending beyond the distal end of the handle, wherein the access needle extends along and between the alignment features and a tissue penetrating tip of the needle is proximal to the ramp, further wherein the slider is positioned along the slot such that a distal tip of the guide element is distal to the ramp and within the recess.

44. The cover of claim 43, wherein proximal movement of the slider moves the distal end of the guide element over the ramp and into a notched distal end portion of the catheter lumen.

45. The cover of any of claims 43 or 44, wherein the handle further comprises a visual or a tactile marking to indicate the position of the distal end of the guide element relative to the notched distal end of the catheter or the tissue penetrating tip of the needle.

46. The cover of any of claims 41 to 45, further comprising the vascular access device of any of claims 1-40.

47. A method of accessing a blood vessel of a patient, comprising:
advancing a tissue penetrating distal tip of an access needle of an intravascular access device in an armed configuration into a blood vessel of the patient;
engaging a guide element with a leg of a slider, the guide element extending through a proximal end of the intravascular access device through a distal tip of a catheter;
advancing the slider distally within a slot that extends longitudinally along the intravascular access device, wherein the slider leg is advanced across a ratchet system on an interior surface of the intravascular access device;
advancing a distal tip of the guide element from within a notch at the distal tip of the catheter into blood vessel distal to the tissue penetrating tip of the access needle;
advancing a distal portion of the catheter hub in the blood vessel along the guide structure; and
retracting a needle carrier coupled to a proximal end of the access needle into the intravascular access device.

48. The method of claim 47, further comprising applying a force to the slider before advancing the slider, wherein the force increases a frictional engagement between the leg of the slider and the guide element.

49. The method of claim 47, further comprising confirming vascular access when blood is observed in a catheter hub coupled to the distal end of the intravascular access handle.

50. The method of claim 47, further comprising uncoupling a catheter hub from a distal end of the intravascular access device handle after the catheter is positioned within the blood vessel.

51. The method of claim 47, further comprising withdrawing the intravascular access device handle proximally along the guide element after the slider is advanced to a distal end of the slot.

52. The method of claim 47, further comprising determining a length of guidewire advanced into the blood vessel based on the distance the slider was advanced over the ratchet system.

53. The method of claim 47, the slider is advanced from a neutral configuration with the leg of the slider contacting a proximal trough of the ratchet system.

54. The method of claim 47, further comprising the ratchet system preventing proximal translation of the slider after the slider has been advanced.

55. The method of claim 47, further comprising advancing the slider leg to contact a proximal end of the needle carrier.

56. The method of any of claims 47-55, further comprising ceasing to perform the step of advancing a tissue penetrating distal tip of an access needle of the intravascular catheter assembly when bleed back is observed in the intravascular catheter assembly.

57. An intravascular access device according to any of claims 1-40, further comprising: a needle cover having a proximal opening sized to engage with a portion of the handle; a lumen in communication with the proximal opening, the lumen sized to receive the needle and catheter; a ramp at a distal position of the lumen adjacent to a recess in the distal most portion of the lumen wherein in use with an IVAD within the needle cover the distal tip of the needle is separated from the guide element by a portion of the ramp and the guide element is in a coiled condition within the recess.

58. The intravascular access device of claim 57, wherein the needle cover and the intravascular device are adapted and configured to interoperate into one or more of a neutral condition, an armed condition, a ready for use condition and a disposal condition.

59. The method of accessing a blood vessel according to any of claims 47-56, further comprising: one or more steps of interaction between the vascular access device and a needle cover so as to render the vascular access device into one or more of a neutral condition, an armed condition, a ready for use condition and a disposal condition.

60. An intravascular access device, comprising:
a handle comprising a proximal end and a distal end, a slot in a surface of the handle having a distal end and a proximal end, and a plurality of ratchet teeth along an interior of the handle;
a slider positioned in the slot, the slider comprising a slider leg extending towards the plurality of ratchet teeth;
a needle carrier having a distal end coupled to an access needle and a proximal end positioned within the handle;
a catheter having a proximal end coupled to a catheter hub, the catheter hub configured to engage the distal end of the handle, the catheter comprising a lumen configured to accommodate the access needle extending therethrough;
a guide element extending from the slider through the catheter lumen, the guide element comprising a distal tip configured to engage a notched distal tip of the catheter, wherein the guide element is configured to be advanced distally from the notched distal tip of the catheter by the slider leg.

61. The device of claim 60, further comprising a dock at the proximal end of the needle carrier adapted to receive the slider leg therein.

62. The device of claim 60, further comprising a channel along the needle carrier adapted to receive the guide element therein.

63. The device of claim 60, wherein a channel adapted to receive the guide element therethrough extends along the needle carrier.

64. The device of claim 60, wherein the access needle comprises a flat surface along the needle exterior.

65. The device of claim 64, wherein the needle carrier comprises a channel along the needle carrier, the channel adapted to receive the guide element therethrough.

66. The device of claim 65, wherein the channel is positioned on the needle carrier at the same clock position as the needle flat surface.
